SEQUENCE LISTING (41 sequences)

SEQ ID NO: 1 - protein, 448 aa, Artificial Sequence
  Anti-C5 (BNJ421) variable heavy chain
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence [sequence omitted]

SEQ ID NO: 3 - protein, 122 aa, Artificial Sequence [sequence omitted]

SEQ ID NO: 4 - protein, 107 aa, Artificial Sequence [sequence omitted]

SEQ ID NO: 5 - protein, 10 aa, Artificial Sequence
  Anti-C5 (BNJ421) variable heavy chain
GHIFSNYWIQ

SEQ ID NO: 6 - protein, 17 aa, Artificial Sequence
  Anti-C5 (BNJ421) variable heavy chain
EILPGSGHTEYTENFKD

SEQ ID NO: 7 - protein, 13 aa, Artificial Sequence
  Anti-C5 (BNJ421) variable heavy chain
YFFGSSPNWYFDV

SEQ ID NO: 8 - protein, 11 aa, Artificial Sequence
  Anti-C5 (BNJ421) variable light chain
GASENIYGALN

SEQ ID NO: 9 - protein, 7 aa, Artificial Sequence
  Anti-C5 (BNJ421) variable light chain
GATNLAD

SEQ ID NO: 10 - protein, 9 aa, Artificial Sequence
  Anti-C5 (BNJ421) variable light chain
QNVLNTPLT

SEQ ID NO: 11 - protein, 326 aa, Artificial Sequence
  Anti-C5 constant heavy chain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 12 - protein, 5 aa, Artificial Sequence
  Anti-C5 (7086) variable heavy chain
SYAIS

SEQ ID NO: 13 - protein, 17 aa, Artificial Sequence
  Anti-C5 (7086) variable heavy chain
GIGPFFGTANYAQKFQG

SEQ ID NO: 14 - protein, 7 aa, Artificial Sequence
  Anti-C5 (7086) variable heavy chain
DTPYFDY

SEQ ID NO: 15 - protein, 11 aa, Artificial Sequence
  Anti-C5 (7086) variable light chain
SGDSIPNYYVY

SEQ ID NO: 16 - protein, 7 aa, Artificial Sequence
  Anti-C5 (7086) variable light chain
DDSNRPS

SEQ ID NO: 17 - protein, 11 aa, Artificial Sequence
  Anti-C5 (7086) variable light chain
QSFDSSLNAEV

SEQ ID NO: 18 - protein, 116 aa, Artificial Sequence
  Anti-C5 (7086) variable heavy chain
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 19 - protein, 108 aa, Artificial Sequence
  Anti-C5 (7086) variable light chain
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 20 - protein, 4 aa, Artificial Sequence
  Anti-C5 (8110) constant heavy chain
NYIS

SEQ ID NO: 21 - protein, 17 aa, Artificial Sequence
  Anti-C5 (8110) variable heavy chain
IIDPDDSYTEYSPSFQG

SEQ ID NO: 22 - protein, 8 aa, Artificial Sequence
  Anti-C5 (8110) variable heavy chain
YEYGGFDI

SEQ ID NO: 23 - protein, 11 aa, Artificial Sequence
  Anti-C5 (8110) variable light chain
SGDNIGNSYVH

SEQ ID NO: 24 - protein, 7 aa, Artificial Sequence
  Anti-C5 (8110) variable light chain
KDNDRPS

SEQ ID NO: 25 - protein, 9 aa, Artificial Sequence
  Anti-C5 (8110) variable light chain
GTYDIESYV

SEQ ID NO: 26 - protein, 116 aa, Artificial Sequence
  Anti-C5 (8110) variable heavy chain
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 27 - protein, 106 aa, Artificial Sequence
  Anti-C5 (8110) variable light chain
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 28 - protein, 6 aa, Artificial Sequence
  Anti-C5 (305LO5) variable heavy chain
SSYYVA

SEQ ID NO: 29 - protein, 17 aa, Artificial Sequence
  Anti-C5 (305LO5) variable heavy chain
AIYTGSGATYKASWAKG

SEQ ID NO: 30 - protein, 13 aa, Artificial Sequence
  Anti-C5 (305LO5) variable heavy chain
DGGYDYPTHAMHY

SEQ ID NO: 31 - protein, 11 aa, Artificial Sequence
  Anti-C5 (305LO5) variable light chain
QASQNIGSSLA

SEQ ID NO: 32 - protein, 7 aa, Artificial Sequence
  Anti-C5 (305LO5) variable light chain
GASKTHS

SEQ ID NO: 33 - protein, 12 aa, Artificial Sequence
  Anti-C5 (305LO5) variable light chain
QSTKVGSSYGNH

SEQ ID NO: 34 - protein, 123 aa, Artificial Sequence
  Anti-C5 (305LO5) variable heavy chain
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 35 - protein, 110 aa, Artificial Sequence
  Anti-C5 (305LO5) variable light chain
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 36 - protein, 451 aa, Artificial Sequence
  Anti-C5 (SKY59) variable heavy chain
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 37 - protein, 217 aa, Artificial Sequence
  Anti-C5 (SKY59) variable light chain
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 120 aa, Artificial Sequence
  Anti-C5 (REGN3918) variable heavy chain
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 39 - protein, 107 aa, Artificial Sequence
  Anti-C5 (REGN3918) variable light chain
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 40 - protein, 447 aa, Artificial Sequence
  Anti-C5 (REGN3918) variable heavy chain
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 214 aa, Artificial Sequence
  Anti-C5 (REGN3918) variable light chain
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0356234 A1
(43) Pub. Date: Nov. 10, 2022

(54) COMPLEMENT INHIBITORS FOR TREATING DRUG-INDUCED COMPLEMENT-MEDIATED RESPONSE

(71) Applicant: Alexion Pharmaceuticals, Inc., Boston, MA (US)

(72) Inventors: Yi Wang, Woodbridge, CT (US); Susan Liu-Chen, Woodbridge, CT (US)

(73) Assignee: Alexion Pharmaceuticals, Inc., Boston, MA (US)

(21) Appl. No.: 17/764,781

(22) PCT Filed: Oct. 1, 2020
(86) PCT No.: PCT/US2020/053673

§ 371 Date: Mar. 29, 2022

Related U.S. Application Data
(60) Provisional application No. 62/909,554, filed on Oct. 2, 2019.

(51) Int. Cl. C07K 16/18; C07K 14/705; C07K 14/47; A61P 37/06

(52) U.S. Cl. CPC C07K 16/18; C07K 14/705; C07K 14/4702; A61P 37/06

(57) ABSTRACT

Disclosed herein are methods and compositions for reducing or eliminating a complement-mediated response in a patient receiving treatment for a disease or disorder wherein one or more therapeutic agents is administered to the patient along with one or more complement inhibitors. Administration of the complement inhibitor along with the therapeutic agent results in a reduced or eliminated complement-mediated response, such as a reduction or elimination of symptoms associated with Complement Activation-Related Pseudoallergy (CARPA) or Cytokine Release Syndrome (CRS).

### CROSS REFERENCE TO RELATED APPLICATIONS

This application claims priority to U.S. Provisional Patent Application No. 62/909,554, filed Oct. 2, 2019, the content of which is hereby incorporated by reference in its entirety.

### REFERENCE TO SEQUENCE LISTING SUBMITTED AS A COMPLIANT ASCII TEXT FILE (.txt)

Pursuant to the EFS-Web legal framework and 37 CFR §§ 1.821-825 (see MPEP § 2442.03(a)), a Sequence Listing in the form of an ASCII-compliant text file (entitled “3000030-013977_Sequence_Listing_ST25.txt” created on 30 Sep. 2020, and 37,235 bytes in size) is submitted concurrently with the instant application, and the entire contents of the Sequence Listing are incorporated herein by reference.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade progresses via the classical pathway, the alternative pathway, or the lectin pathway. These pathways share many components, and while they differ in their initial steps, they converge and share the same “terminal complement” components (C5 through C9) responsible for the activation and destruction of target cells.

The classical pathway (CP) is typically initiated by antibody recognition of, and binding to, an antigenic site on a target cell. The alternative pathway (AP) can be antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease to yield C3a and C3b. Other pathways activating complement attack can act later in the sequence of events leading to various aspects of complement function.

The complement system is comprised of several small proteins organized into a biochemical cascade serving to assist the immune system in the clearance of pathogens. The complement proteins circulate in the blood as inactive precursors and, when stimulated by one of several triggers, proteases in the system cleave specific proteins to release cytokines and initiate an amplifying cascade of further cleavages. Cytokine release syndrome (“CRS”) is a potentially life threatening systemic inflammatory reaction that is observed after infusion of agents targeting different immune effectors. Affected patients mostly develop feller, chills, hypotension, and tachycardia during or immediately after drug administration. Furthermore, the syndrome may cause a broad spectrum of constitutional and organ-related disorders, as well as blood test abnormalities. CRS is driven by an increase of inflammatory cytokines that are released after the activation and cytotoxic damage of monocytes, macrophages, and different lymphocyte populations

Complement Activation Related Pseudo Allergy (“CARPA”) is a serious condition commonly following administration of certain types of drugs and nanotechnology-based combination products. While CARPA symptoms are similar to that olanaphylaxis, the mechanism behind this pathology does not involve IgE and is mediated by the complement system.

CARPA and CRS are serious issues that present especially during administration of other therapeutics, there is a need to identify materials and methods for suppressing CARPA and CRS.

### SUMMARY

Provided herein are methods and compositions for reducing or eliminating a complement-mediated response in a patient receiving treatment for a disease or disorder comprising administering to the patient a composition comprising one or more therapeutic agents, wherein the composition is capable of local or systemic activation of a complement system; and administering to the patient one or more complement inhibitors, optionally a short-acting complement inhibitor. In various embodiments, the reduced or eliminated complement-mediated response is a reduction or elimination of symptoms associated with Complement Activation-Related Pseudoallergy (CARPA) or Cytokine Release Syndrome (CRS).

In various embodiments, the compositions and methods comprise a therapeutic agent selected from gene therapy, mRNA therapy, antibody therapy, or a cell therapy. In other embodiments, the one or more therapeutic agents is delivered to the patient utilizing a lipid drug delivery system, optionally wherein the therapeutic is encapsulated in a lipid nanoparticle, nanostructured lipid carrier, a lipid drug conjugate-nanoparticle, a liposome, a transfersome, an ethosonte, liposphere, a niosome, a cubosome, a virosome, as iscom, a mmoemulsion, or a phytosome.

In certain embodiments, the one or more complement inhibitors inhibits an enzymatic activity of a soluble complement protein in the patient, for example, cleavage of a complement component selected from the group consisting of: C5, C6, C7, CS, C9, factor D, and factor B.

The therapeutic agent and the complement inhibitor can be administered concurrently or sequentially, and can be administered systemically or locally to an extravascular location such as subcutaneous, intraperitoneal, intramuscular, intra-articular, intra-synovial, intrasternal, intrathecal, intrahepatic, intralesional, intracranial, intraventricular, oral, pulmonary, topical, rectal, nasal, buccal, vaginal, intratumoral, and intradermal.

In some of the foregoing embodiments, the one or more complement inhibitors is administered in an amount sufficient to produce a clinically significant reduction in severity of at least one symptom of CARPA or CRS, as compared to, when the one or more complement inhibitors is not administered with the one or more therapeutic agents.

Also provided are pharmaceutical compositions comprising the complement inhibitor, optionally formulated for systemic delivery of for delivery to a specific extravascular location.

### BRIEF DESCRIPTION OF THE DRAWINGS

FIG. 1 is a bar graph showing the cytokine response induced by various injected agents (PBS buffer control, luciferase mRNA, human erythropoietin (hEPO) mRNA and hEPO protein). These data show that a single dose mRNA administration elicits a cytokine response (IL 6, KC/GRO and TNF-alpha) at 2 and. 6 hours—the response essentially returning to baseline by 24 hours.

FIG. 2A-2D is a series of bar graphs showing an induced cytokine response when LNP formulated mRNA or protein were administered. These data indicate a single dose of LUNAR LNP mRNA (“formulated mRNA”) elicits dose dependent cytokine response at 2 and 6 hours for IL 6, KC/GRO, TNF-alpha, and IL 12; the cytokine response is resolved by 24 hours.

FIG. 3A-3D is a series of bar graphs showing an induced cytokine response when LNP formulated m.RNA or protein were administered. After the sixth weekly dosing, plasma IL 6, TNF-alpha, IL 10 and. KC were elevated at 2 hours and resolved by 24 hours.

FIG. 4 shows that BB5.1 and scFV in ibit TNF-alpha response at 2 h when co-dosed with formulated mRNA, but TT 30 does not.

FIG. 5 shows that TT30 inhibits TNF-alpha response at 6 h when co-dosed with formulated mRNA.

FIG. 6 shows that plasma TNF-alpha is resolved by 24 hours when co-dosed with formulated mrRNA.

### DETAILED DESCRIPTION

### I. Overview

Provided herein are methods and compositions for reducing or eliminating a complement-mediated response in a subject receiving treatment for a disease or disorder wherein the subject (e.g., patient) is administered one or more therapeutic agents capable of local or systemic activation of a complement system in combination with one or more complement inhibitors. A concise summary of the biologic activities associated with complement activation is provided, thr example, in The Merck Manual, 16th Edition. A “subject,” as used herein, can be any mammal. A subject can be, for example, a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. In some embodiments, the subject is an infant (e.g., a human infant). As used herein, a subject “in need of prevention,” “in need of treatment,” or “in need thereof,” refers to one, who by the judgment of an appropriate medical practitioner (e.g., a doctor, a nurse, or a nurse practitioner in the case of humans a veterinarian in the case of non-human mammals), would reasonably benefit from a given treatment. As described herein, a subject in need of a particular therapeutic agent to treat a disease or disorder, would also be in need of treatment with a complement inhibitor to suppress the complement-mediated effect (e.g., cytokine release syndrome or CARPA) produced by the primary therapeutic agent.

The complement system is comprised of several small proteins organized into a biochemical cascade serving to assist the immune system in the clearance of pathoeens. The complement proteins circulate in the blood as inactive precursors. When stimulated by one of several triggers, proteases in the system cleave specific proteins to release cytokines and initiate an amplifying cascade of further cleavages. Cytokine release syndrome (“CRS”) is a potentially life threatening systemic inflammatory reaction that is observed after infusion of aeents targeting different immune effectors. Affected patients mostly develop fever, chills, hypotension, and tachycardia during or immediately after drug administration. Furthermore, the syndrome may cause a broad spectrum of constitutional and organ-related disorders, as well as blood test abnormalities. CRS is driven by an increase of inflammatory cytokines that are released after the activation and cytotoxic damage of monocytes, macrophages, and different lymphocyte populations (Lee et al. (2014) Blood, 124(2): 188-95).

CARPA and CRS are common dose-limiting toxicities for particular types of drug products including therapeutic oligonucleotides (Shen, L. et al., Nucleic Acid Ther., 26:236-49, 2016; Shen, L. et al., J. Pharmacol. Exp. Ther., 351:709-17, 2014; Henry, S. et al., Int. Immunopharmacol., 2:1657-66, 2002) and PEGylated liposomal formulations of small molecules (Rampton, D. et al., Haematologica, 99:1671-6, 2014; Szebeni, J., Mol. Immunol., 61:163-73, 2014; and Vonarbourg, A. et al., J. Biomed. Mater. Res. A, 78:620-8, 2006).

Inhibition of complement (e.g., inhibition of terminal complement formation, C5 cleavage, or complement activation) has been demonstrated to be effective in treating several complement-associated disorders both in animal models and in humans (Rother, R. et al., Nat. Biotechnol., 25:1256-64, 2007; Wang, Y et al., Proc. Natl. Acad. Sci. USA. 93:8563-8, 1996; Wang, Y. et al., Proc. Natl. Acad. Sci. USA, 92:8955-9, 1995; Rinder, C. et al., J. Clin. Invest., 96:1564-72, 1995; Kroshus, T. et al., Transplantation, 60:1194-202, 1995; Homeister, J. et al., J. Immuunol., 150:1055-64, 1993; Weisman, H. et al., Science, 249:146-51, 1990; Amsterdam, E. et al., Am. J. Physiol., 268:H448-57, 1995; and Rabinovici, R. et al., J. Immunol., 149:1744-50, 1992).

In various embodiments, the complement inhibitor is an agent that inhibits the enzymatic activity of a complement component. A “complement component” or “complement protein” is a molecule that is involved in activation of the complement system or participates in one or more complement-mediated activities. Components of the classical complement pathway include, C1q, C1r, C1s, C2, C3, C4, C5, C6, C7, C8, C9 and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1 and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will he appreciated that the term “complement component” is not intended to include those molecules and molecular structures that serve as “triggers” for complement activation, e.g., antigen-antibody complexes, foreign structures found on microbial or artificial surfaces, etc.

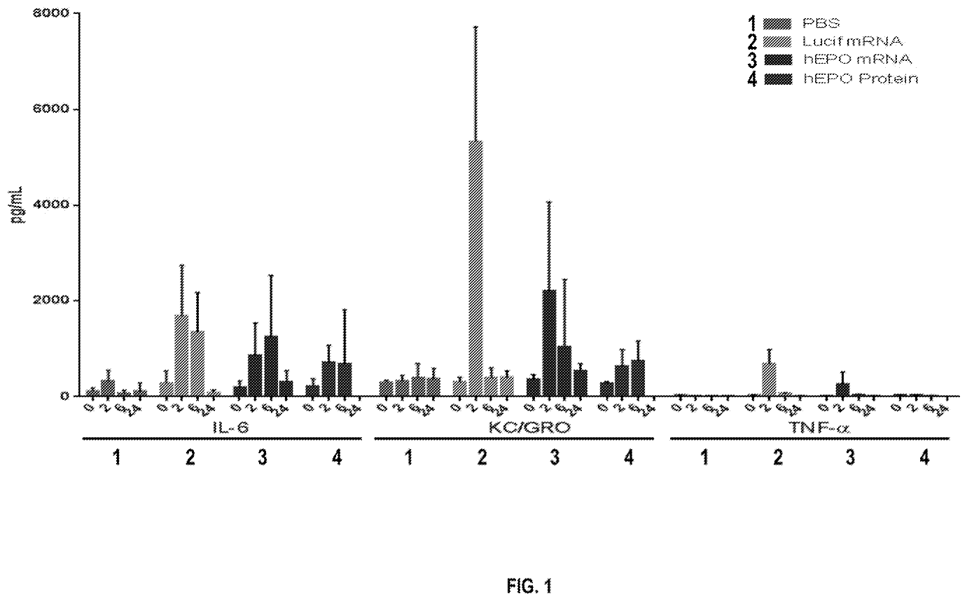

In various embodiments, the complement inhibitor is a short-acting inhibitor. By “short-acting inhibitor” is intended that the agent inhibits the enzymatic activity of a complement component for 20 minutes to one hour, or from 20 minutes to 2 hours, from 30 minutes to 3 hours, from 1 hour to 2 hours, from 1 hour to 4 hours, from 20 minutes to 4 hours, from about 20 minutes to about 6 hours, from about 20 minutes to about 8 hours, from about 20 minutes to about 10 hours, from about 20 minutes to about 12 hours, or any increment thereof. Examples of short-acting complement inhibitors include, but are not limited to, the CR2-fH fusion protein TT30 (Risitano, A. et al, Blood, 119:6307-16, 2012; Rohrer B. et al., Adv. Exp. Med. Biol., 703:137-49, 2010; Rohrer, B. et al., Invest. Ophthalmol. Vis. Sci., 50:3056-64, 2009; WO 2007/149567). In some embodiments, the activity of the complement inhibitor is transitory, i.e., the inhibition of complement activation is resolved after a period of about 30 minutes, about 1 hour, about 2 hours, about 3 hours, about 4 hours, about 6 hours, about 8 hours, about 10 hours, or about 12 hours following administration of the complement inhibitor, particularly at the site of administration of the inhibitor. See, for example, FIGS. 1-3, which demonstrate that the levels of various cytokines return to baseline 24 hours after administration of a therapeutic mRNA in combination with a complement inhibitor. In the present context, “resolved” means that a measured cytokine level, measured after therapeutic administration, has returned to a level that is at or near (e.g., within about 5% to about 10%) of a level that was measured, before therapeutic administration, for that cytokine (i.e., a “baseline level”).

In some embodiments, the therapeutic agent is capable of systemic activation of the complement system, and the therapeutic agent and complement inhibitor are administered systemically. “Systemic complement activation” is complement activation that occurs in the blood, plasma or serum and/or involves activation of systemic complement proteins at many locations throughout the body, affecting many body tissues, systems, or organs. “Systemic administration” and like terms are used herein consistently with their usage in the art to refer to administration of an agent such that the agent becomes widely distributed in the body in significant amounts and has a biological effect, e.g., its desired effect, in the blood and/or reaches its desired site of action via the vascular system. Typical systemic routes of administration include administration by (i) introducing the agent directly into the vascular system or (ii) oral, pulmonary, or intramuscular administration wherein the agent is absorbed, enters the vascular system, and is carried to one or more desired site(s) of action via the blood.

A variety of different complement inhibitors are useful for the methods described herein. Such complement inhibitors fall into a number of compound classes including peptides, polypeptides, antibodies, small molecules and nucleic acids. Complement inhibitors include antagonists of one or more proteins in the classical, alternative and/or lectin pathway. In certain embodiments, the complement inhibitor inhibits an enzymatic activity of a complement protein. The enzymatic activity may be proteolytic activity, such as ability to cleave another complement protein.

In various aspects, complement-inhibiting compounds can also comprise either naturally occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation.

Other compounds, e.g., polypeptides, small molecules, monoclonal antibodies, aptamers, etc., that bind to complement pathway receptors are of use in certain embodiments (e.g., U.S. Pat. No. 5,942,405 discloses C3aR antagonists. U.S. Pat. Pub. No. 20030191084 discloses aptamers that bind to C1q, C3 and C5).

A. Compounds that Inhibit C5 Activation or Activity

In certain embodiments the complement inhibitor inhibits activation of C5, thereby reducing, suppressing and/or eliminating the complement-mediated effects CSR or CARPA) that occur during therapeutic administration of certain therapeutics (e.g., particle or nanoparticle encapsulated therapeutics). Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

A complement inhibitor suitable for use in reducing, suppressing and/or eliminating the complement-mediated effects (e.g., CSR or CARPA) that occur during therapeutic administration of certain therapeutics (e.g., particle or nanoparticle encapsulated therapeutics) may bind to C5. Exemplary agents include antibodies, antibody fragments, polypeptides, small molecules, and aptamers. Exemplary antibodies are described in U.S. Pat. No. 6,534,058 and in Wang, et al., Proc. Natl. Acad. Sci, USA, 92;8955-8959, 1995. Exemplary compounds that bind to and inhibit C5 are described in U.S. Pat. Pub, Nos. 20050090448 and 20060115476. In certain embodiments the complement inhibitor is an antibody, small molecule, aptamer, or polypeptide that binds to substantially the same binding site on C5 as an antibody described in U.S. Pat. No. 6,534,058 ora peptide described in U.S. Ser. No. 10/937,912, U.S. Pat. Pub. No. 20060105980 discloses aptamers that bind to and inhibit C5. RNAi agents that inhibit local expression of C5 or CSR can also be used in the methods described herein.

In other embodiments the agent is an antagonist of a C5a receptor (C5aR).

C5a is cleaved from the alpha chain of C5 by either alternative or classical C5 convertase. The cleavage site for convertase action is at, or immediately adjacent to, amino acid residue 733 of the alpha chain of C5a. A compound that would bind at, or adjacent to, this cleavage site would have the potential to block access of the C5 convertase enzymes to the cleavaae site and thereby act as a complement inhibitor. A compound that binds to C5 at a site distal to the cleavage site could also have the potential to block C5 cleavage, for example, by way of steric hindrance-mediated inhibition of the interaction between C5 and the C5 convertase. Exemplary C5a receptor antagonists include a variety of small cyclic'peptides such as those described in U.S. Pat. No. 6,821,950; U.S. Ser. No. 11/375,587; and/or PCT/US06/08960 (WO2006/099130), or the monoclonal antibody BB5.1 (Frei Y. et al., Mol. Cell. Probes, 1:141-9, 1987), the single chain variable fragment (scFV) of BB5.1, or the anti-BB5.1 Fab (Peng et al., J Clin Invest, 115(6)1590-1600, 2005), which prevent the formation or C5a and C5b.

In certain embodiments, the complement inhibitor comprises an anti-C5 antibody. Anti-C5 antibodies (or VH/VL domains derived therefrom) suitable for use herein can be identified using methods known in the art. Alternatively, art recognized anti-C5 antibodies can be used. Antibodies that compete with any of these art recognized antibodies for binding to C5 also can be used.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia, C. et al. (Nature, 342:877 83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat Chothia combined definition. In such embodiments, these regions can be referred to as “combined Kabat Chothia CDRs” (Thomas, T. et al., Mol. Immunol., 33:1389 401, 1996) exemplifies the identification of CDR boundaries according to Kabat and Chothia definitions.

Another exemplary anti-C5 antibody is antibody BNJ421comprising heavy and light chains having the sequences shown in SEQ ID NOs:1 and 2, respectively, or antigen binding fragments and variants thereof. BNJ421 is described in PCT/US2015/019225 and U.S. Pat. No. 9,079,949, the teachings of which are incorporated herein by reference. The anti-C5 antibody can comprise, for example, the heavy and light chain CDRs or variable regions of BNJ421, e.g., CDR1, CDR2 and CDR3 of the VH region of BNJ421 having the sequence set forth in SEQ ID NO:3, and CDR1, CDR2 and CDR3 of the VL region of BNJ421 having the sequence set forth in SEQ ID NO:4. The anti-C5 antibody can comprise, for example, heavy chain CDR1, CDR2 and CDR3 domains having the sequences set forth in SEQ ID NOs:5, 6, and 7, respectively, and light chain CDR1, CDR2 and CDR3 domains having the sequences set forth in SEQ ID NOs:8, 9 and 10, respectively BNJ421 comprises VH and VL regions having the amino acid sequences forth SEQ ID NO:3 and SEQ ID NO:4, respectively.

The anti-C5 antibody can comprise, for example, a heavy chain constrant region as set forth in SEQ ID NO:11.

The anti-C5 antibody can comprise, for example, a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

Another exemplary anti-C5 antibody is the 7086 antibody described in U.S. Pat. Nos. 8,241,628 and 8,883,158. The anti-C5 antibody can comprise, for example, the heavy and light chain CDRs or variable regions of tbe 7086 antibody. The anti-C5 antibody can comprise, for example, comprises heavy chain CDR1, CDR2 and CDR3 domains having the sequences set forth in SEQ ID NOs: 12, 13, and 14, respectively, and light chain CDR1, CDR2 and CDR3 domains having the sequences set forth SEQ ID NOs: 15, 16, and 17, respectively. The anti-C5 antibody can comprise, for example, the VH region of the 7086 antibody having the sequence set forth in SEQ ID NO:18, and the VL region of the 7086 antibody having the sequence set forth in SEQ ID NO:19.

Another exemplary anti-C5 antibody is the 8110 antibody also described in U.S. Pat. Nos. 8,241,628 and 8,883,158, The anti-C5 antibody can comprise, for example, the heavy and light chain CDRs or variable regions of the 8110 antibody. The anti-C5 antibody can comprise, for example, heavy chain CDR1, CDR2 and CDR3 domains having the sequences set forth in SEQ ID NOs: 20, 21, and 22, respectively, and light chain CDR1, CDR2 and CDR3 domains having the sequences set forth in SEQ ID NOs: 23, 24, and 25, respectively. The anti-C5 antibody can comprise, for example, the VH region of the 8110 antibody having the sequence set forth in SEQ ID NO:26, and the VL region of the 8110 antibody having the sequence set forth in SEQ ID NO:27.

Another exemplary anti-C5 antibody is the 305LO5 antibody described in US2016/0176954A1. The anti-C5 antibody can comprise, for example, the heavy and light chain CDRs or variable regions of the 305LO5 antibody. The anti-C5 antibody can comprise, for example, heavy chain CDR1, CDR2 and CDR3 domains having the sequences set forth in SEQ ID NOs:28, 29 and 30, respectively, and light chain CDR1, CDR2 and CDR3 domains having the sequences set forth in SEQ ID NOs:31, 32, and 33, respectively. In another embodiment, the antibody comprises the VH region of the 305LO5 antibody having the sequence set forth in SEQ ID NO:34, and the VL region of the 305LO5 antibody having the sequence set forth in SEQ ID NO: 35.

Another exemplary anti-C5 antibody is the SKY59 antibody (Fukuzawa, T. et al., Sci. Rep., 7:1080, 2017), The anti-C5 antibody can comprise, for example, the heavy and light chain CDRs or variable regions of the SKY59 antibody. The anti-C5 antibody can comprise, for example, a heavy chain comprising SEQ ID NO:36 and a light chain comprising SEQ ID NO: 37.

Another exemplary anti-C5 antibody is the REGN3918 antibody (also known as H4H12166PP) described in US20170355757. The anti-C5 antibody can comprise, for example, a heavy chain variable region comprising SEQ ID NO:38 and a light chain variable region comprising SEO ID NO:39, or a heavy chain comprising SEQ ID NO:40 and a light chain comprising SEQ ID NO:41.

In another embodiment, the antibody competes for binding with, and/or binds to the same epitope oar C5 as, the above-mentioned antibodies (e.g., 7086 antibody, 8110 antibody, 305LO5 antibody, SKY59 antibody, or REGN3918 antibody). The anti-C5 antibody can have, for example, at least about 90% variable region amino acid sequence identity with the above-mentioned antibodies (e.g., at least about 90%, 91%, 92%, 93%, 94%, 95%, 96%, 97%, 98% or 99% variable region identity).

An anti-C5 antibody described herein can, in some soare embodiments, comprise a variant human Fc constant region that binds to human neonatal Fc receptor (FcRn) with greater affinity than that of the native human Fc constant region from which the variant human Fc constant region was derived. The Fc constant region can comprise, tbr example, one or more (e.g., two, three, four, five, six, seven, or eight or more) amino acid substitutions relative to the native human Fc constant region from which the vaaiant human Fc constant region was derived. The substitutions, for example, can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples (PCT/US2015/019225 and U.S. Pat. No. 9,079,949 the disclosures of each of which are incorporated herein by reference in their entirety).

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514 24, 2006); (2) the M428L or T250Q/M428L substitutions (Hinton, P. et al., J. Biol. Chem., 279:6213 6, 2004; Hinton, P. et al., J. Immunol., 176:346 56, 2006); and (3) the N434A or T307/E380A/N434A substitutions (Petkova, S. et al., Int. Immunol., 18:1759 69, 2006). Additional substitution pairings, e.g., P2571/Q3 11I, P257I/N434H, and D376V/N434H, have also been described (Datta-Mannan, A. et al., J. Biol. Chem., 282:1709 17, 2007). The entire teachings of each of the cited references are hereby incorporated by reference.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three or two) amino arid substitutions, insertions or deletions relative to the native constant region from which it was derived, in some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376 the disclosure of which is incorporated herein by reference in its entirety.

In some embodiments the precise location of these mutations may be shifted from the native human Fc constant region position due to antibody engineering. The 428L/434S double substitution when used in a IgG2/4 chimeric Fc, for example, may correspond to 429L and 435S as in the M429L and N435S variants described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan or tyrosine for threonine at position 250; phenylalanine, tryptophan or tyrosine for methionine at position 252; threonine for serine at position 254; glutamic acid for arginine at position 255; aspartic acid, glutamic acid or glutamine for threonine at position 256; alanine, glycine, isoleucine, leucine, methionine, asparagine, serine, threonine or valine for proline at position 257; histidine for glutamic acid at position 258; alanine for aspartic acid at position 265; phenylalanine for aspartic acid at position 270; alanine or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan or tyrosine for threonine at position 307; alanine, phenylalanine, isoleucine, leucine, methionine, proline, glutamine or threonine for valine at position 308; alanine, aspartic acid, glutamic acid, proline or arginine for leucine or valine at position 309; alanine, histidine or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan or tyrosine for methionine at position 428; lysine for histidine at position 433; alanine, phenylalanine, histidine, serine, tryptophan or tyrosine for asparagine at position 434; and histidine for tyrosine or phenylalanine at position 436, all in EU numbering.

In one embodiment, the antibody binds to C5 at pH 7.4 and 25oC (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3 or 0.2) nM.

In other embodiments, the [(K_{D} of the antibody for C5 at pH 6.0 at 25° C.)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

B. Compounds that Inhibit Factor B Activation or Activity

In certain embodiments the complement inhibitor inhibits activation of factor B. The complement inhibitor can bind to factor B, for example, thereby inhibiting activation. Exemplary agents include antibodies, antibody fragments, peptides, small molecules, and aptamers. Exemplary antibodies that inhibit factor B are described in U.S. Pat. Pub, No. 20050260198. In certain embodiments the isolated antibody or antigen-binding fragment selectively binds to factor B within the third short consensus repeat (SCR) domain. In certain embodiments the antibody prevents formation of a C3bBb complex. In certain embodiments the antibody or antigen-binding fragment prevents or inhibits cleavage of factor B by factor D. In certain embodiments the complement inhibitor is an antibody, small molecule, aptamer, or polypeptide that binds to substantially the same binding site on factor B as an antibody described in U.S. Pat. Pub. No. 20050260198, or is an RNAi agent that inhibits local expression of factor B. Peptides that bind to and inhibit factor B can be identified using methods known in the art.

C. Compounds that Inhibit Factor D Activity

In certain embodiments the complement inhibitor inhibits factor D. The complement inhibitor may bind to factor D, for example, thereby inhibiting factor D. Exemplary agents include antibodies, antibody fragments, peptides, small molecules, and aptamers. While factor D has been suggested as a desirable target for systemic complement inhibition as a result of its relatively low serum concentration and ability to inhibit alternative pathway activation, the present disclosure is directed to the therapeutic potential of locally administered agents that inhibit factor D. Exemplary antibodies that inhibit factor D are described in U.S. Pat. No. 7,112,327. In certain embodiments the complement inhibitor is an antibody, small molecule, aptamer, or polypeptide that binds to substantially the same binding site on factor D as an antibody described in U.S. Pat. No. 7,112,327. Exemplary polypeptides that inhibit alternative pathway activation and are believed to inhibit factor D are disclosed in U.S. Pub. No. 20040038869. Peptides that bind to and inhibit factor D can be identified using methods known in the art,

### D. Multimodal Complement Inhibitors

The complement inhibitor useful in the methods described herein can bind to more than one complement protein and/or inhibit more than one step in a complement activation pathway. Such complement inhibitors are referred to herein as “multimodal.”

The complement inhibitor can be, for example, a virus complement control protein (VCCP) (U.S. Ser. No. 11/247,886 and PCT/US2005/36547, filed Oct. 8, 2005). Poxviruses and herpesviruses are families of large, complex viruses with a linear double-stranded DNA genome. Certain of these viruses encode immunomodulatory proteins that are believed to play a role in pathogenesis by subverting one or more aspects of the normal immune response and/or fostering development of a more favorable environment in the host organism (Kotwal, G., Immunol. Today, 21, 242-8, 2000). Among these are VCCPs. Poxvirus complement control proteins are members of the complement control protein (CCP) superfamily and typically contain four SCR modules. These proteins have features that make them advantageous for local complement inhibition. In certain embodiments the VCCP is a poxvirus complement control protein (PVCCP). The PVCCP can comprise a sequence encoded by, e.g., vaccinia virus, variola major virus, variola minor virus, cowpox virus, monkeypox virus, ectromelia virus, rabbitpox virus, myxoma virus, Yaba-like disease virus, or swinepox virus. In other embodiments the VCCP is a herpesvirus complement control protein (HVCCP). The HVCCP can comprise a sequence encoded by a Macaca fuscata rhadinovirus, cercopithecine herpesvirus 17, or human herpes virus 8. In other embodiments the HVCCP comprises a sequence encoded by herpes simplex virus saimiri ORF 4 or ORF 15 (Albrecht, J. & Fleckenstein, B., J. Virol., 66:3937-40, 1992; Albrecht, J. et al., Virology, 190:527-30, 1992).

The VCCP may inhibit the classical complement pathway, the alternate complement pathway, the lectin pathway, or any two or more of these. The VCCP, e.g., a PVCCP, can bind to C3b, C4b, or both, for example. The PVCCP can comprise one or more putative heparin binding sites (K/R-X-K/R) and/or possesses a overall postive charge. In some embodiaraents, the PVCCP comprises at least 3 SCR modules (e.g., modules 1-3), e.g., 4 SCR modules. The PVCCP protein can be a precursor of a mature PVCCP (i.e., can include a signal sequence that is normally cleaved off when the protein is expressed in virus-infected cells) or can be a mature form (i.e., lacking the signal sequence).

Vaccinia complement control protein (VCP) is a virus-encoded protein secreted from vaccinia infected cells (U.S. Pat. Nos. 5,157,110 and 6,140,472; Kotwal, G. & Moss, B., Nature, 355:176-8, 1988). VCP has been shown to inhibit the classical pathway of complement activation via its ability to bind to C3 and C4 and act as a cofactor for factor I mediated cleavage of these components as well as promoting decay of existing convertase (Kotwal, a et al., Science, 250:827-30, 1990; McKenzie, R. et al., J. Infect. Dis., 166:1245-50, 1992). It has also been shown to inhibit the alternative pathway by causing cleavage of C3b into iC3b and thereby preventing the formation of the alternative pathway C3 convertase (Sahu, A. et al., J. Immunol., 160, 5596-604, 1998). VCP thus blocks complement activation at multiple steps and reduces levels of the proinflammatory chemotactic factors C3a, C4a, and C5a.

Variola virus major and minor encode proteins that are highly homologous to VCP and are referred to as smallpox inhibitor of complement enzymes (SPICE) (Rosengard, A. et al., Proc. Natl. Acad. Sci. USA, 99:8808-13, 2002; U.S. Pat. No. 6,551,595). SPICE from various variola strains sequenced to date differs from VCP by about 5% (e.g., about 11 amino acid differences). Similarly to VCP, SPICE binds to C3b and C4b and causes their degradation, acting as a cofactor for factor I. However, SPICE degrades C3b approximately 100 times as fast as VCP and degrades C4b approximately 6 times as fast a5 VCP. SPICE or any of the portions thereof that inhibit complement activation, e.g., SPICE and SPICE-related polypeptides containing four SCRs, can be used in the methods described herein.

Complement control proteins from cowpox virus (referred to as inflammation modulatory protein, IMP) and monkeypox virus (referred to herein as monkeypox virus complement control protein, MCP) have also been identified (Miller, C. et al., Virology, 229:126-33, 1997; Uvarova, E. & Shchelkunov, S., Virus Res., 81:39-45, 2001) and can be used in the methods described herein.

In addition to VCCPs, a number of other viral proteins exist that interfere with one or more steps in a complement pathway and can be used in the methods described herein. Certain of these proteins do not necessarily display clear homology to cellular complement regulators known to date. For example, HSV-1, HSV-2, VZV, PRV, BHV-1, EHV-1, and EHV-4 all encode versions of a conserved glycoprotein known as gC (Schreurs, C. et al., J. Virol., 62:2251-7, 1988: Mettenleiter, T. et al., J. Virol., 64:278-86, 1990; Herold, B. et al., J. Virol., 65:1090-8, 1991). With the exception of VZV, the gC protein encoded by these viruses binds to C3b (Friedman, H. et al., Nature, 309:633-5, 1984; Huemer, H. et al., Virus Res., 23:271-80, 1992) gC1 (from HSV-1) accelerates decay of the classical pathway C3 convertase and inhibits binding of properdin and C5 to C3. Purified EBV virions possess an activity that accelerates decay of the alternative pathway C3 convertase and serves as a cofactor for the complement regulatory protein factor I (Mold, C. et al., J. Exp. Med., 168:949-69, 1988). The foregoing proteins are referred to collectively as virus complement interfering proteins (VCIPs). By any of a variety of means, such as interfering with one or more steps of complement activation, accelerating decay of a complement component, and/or enhancing activity of a complement regulatory protein, these VCIPs are said to inhibit complement. Any of these proteins, or derivatives thereof, e.g., fragments or variants thereof, can be used as a therapeutic agent in the methods described herein.

### E. Additional Complement Inhibiting Agents, Mixtures, and Modifications

A variety of other complement inhibitors can be used in various embodiments of the methods described herein. In some embodiments, the complement inhibitor is a naturally occurring mammalian complement regulatory protein or a fragment or derivative thereof. The complement regulatory protein can be, for example, CR1, DAF, MCP, CFH or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can be used. The compounds known as TP10 or TP20 (Avant Therapeutics), for example, can be used. C1 inhibitor (C1-INH) is also of use. In some embodiments a soluble complement control protein, e.g., CFH, is used. In some embodiments, the polypeptide is modified to increase its solubility.

Inhibitors of C1s, are of use (e.g., U.S. Pat. No. 6,515,092 describes compounds (furanyl and thienyl amidines, heterocyclic amidines, and guanidines) that inhibit C1s; U.S. Pat. Nos. 6,515,002 and 7,138,530 describe heterocyclic amidines that inhibit C1s; U.S. Pat. No. 7,049,282 describes peptides that inhibit classical pathway activation; U.S. Pat. No. 7,041,796 discloses C3b/C4b Complement Receptor-like molecules and uses thereof to inhibit complement activation; U.S. Pat. No. 6,998,468 discloses anti-C2/C2a infra/inns of complement activation; U.S. Pat. No. 6,676,943 discloses human complement C3-degrading protein from Streptococcus pneumoniae).

Combination therapy using two or more complement inhibitors is encompassed in the methods described herein. The two or more complement inhibitors may be provided in the same composition. In certain embodiments the complement inhibitors bind to two or more different complement components. In certain embodiments the complement inhibitors bind to two or more different soluble complement proteins. In certain embodiments the complement inhibitors inhibit activation or activity of at least two complement proteins selected from C3, C5, C6, C7, C8, C9, factor B, and factor D.

Complement inhibitors, optionally linked to a binding moiety, can be modified by addition of a molecule such as, for example, polyethylene glycol (PEG) or similar molecules to stabilize the compound, reduce its immunogenicity, increase its lifetime in the body, increase or decrease its solubility, and/or increase its resistance to degradation. Methods for pegylation are well known in the art (Veronese, F. & Harris, J., Adv. Drug Deliv. Rev., 54;453-6, 2002; Davis, F., Adv. Drug Deliv. Rev., 54:457-8, 2002; Wang, V. et al., Adv. Drug Deliv. Rev., 54:547-70, 2002). A wide variety of polymers such as PEGs and modified PEGs, including derivatized PEGs to which polypeptides, can conveniently be attached are described in Nektar Advanced Pegylation 2005-2006 Product Catalog, Nektar Therapeutics, San Carlos, Calif., which also provides details of appropriate conjugation procedures. Conjugation to or binding to albumin also increase the serum half-life of a complement inhibitor.

### II. Producing Complement Inhibitors

In general, the complement inhibitors are manufactured using standard methods known in the art and suitable for compounds of that class. Peptides may be manufactured using standard ol phase peptide synthesis techniques. Polypeptides may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or methods involving chemical ligation of synthesized peptides. Recombinant polypeptides may be produced using standard recombinant nucleic acid techniques as described, e.g., in U.S. Ser. No. 11/247,886 and PCT/US2005/36547 (WO2006042252) and expression systems. See, e.g., Hardin, C., et al., (Eds.), “Cloning, Gene Expression and Protein Purification: Experimental Procedures and Process Rationale”, Oxford University Press, Oxford, 2001. Activity of certain polypeptides is at least partly dependent on their glycosylation state. It may be desirable to produce such pol peptides in systems that provide for glycosylation similar or substantially identical to that found in mammals, e.g., humans. Mammalian expression systems or modified lower eukaryotic expression systems (e.g, fungal expression systems) that provide for mammalian-like glycosylation can be used. See, e.g., U.S. Pub. Nos. 20060177898 and 20070184063. Antibodies, e.g., monoclonal antibodies, may be harvested from hybridomas or produced using recombinant methods as known in the art. Chemical modifications such as pegylation may be performed using standard methods.

### III. Measuring Complement Inhibition

Any suitable method can be used for assessing the ability of an agent or composition containing the agent to inhibit complement activation (or any other relevant properties). A number of in vitro assays can be used. The ability of an agent to inhibit the classical or alternative complement pathway, for example, can be assessed by measuring complement-mediated hemolysis of erythrocytes (e.g., antibody-sensitized or unsensitized rabbit or sheep erythrocytes), by human serum or a set of complement components in the presence or absence of the agent. The ability of an agent to bind to one or more complement components such as C3, C5, C6, C7, C8, C9, factor B or factor D can be assessed using, for example, isothermal titration calorimetry or other methods suitable for performing in liquid phase. The ability of an agent to bind to a complement component can be measured, for example, using an ELISA assay. Other methods of use include surface plasmon resonance, equilibrium dialysis, etc.

Methods for measuring systemic or local complement activation taking place in vitro or in vivo and for determining the ability of a complement inhibitor to inhibit such activation are known in the art. Measurement of complement activation products such as C3a, C5a, C3bBb, C5b-9, etc., for example, provides an indication of the extent of complement activation. A decrease in the amount of such products indicates inhibition of complement activation. In some embodiments a ratio between an active cleavage product and its inactive desArg form is measured (e.g., C3a/C3adesArg). One of skill in the art can distinguish between classical, alternative, and lectin pathway activation by appropriate selection of the complement activation product(s) measured and/or appropriate activators of complement such as zymosan, lipopolysaccharide, immune complexes, etc. Other methods involve measuring complement-mediated hemolysis of red blood cells as a result of terminal complex formation.

Complement activation in vivo and/or its inhibition by a complement inhibitor, can be measured in an appropriate biological sample. Systemic complement activation and/or its inhibition by a complement inhibitor, can be measured in a blood sample, for example. Serial measurements beginning before administration of a complement inhibitor provide an indication of the extent to which the complement inhibitor inhibits complement activation and the time course and duration of the inhibition. It will be appreciated that a decrease in activation products may only become apparent once activation products present prior to administration of the complement inhibitor bave been degraded or cleared.

The in vivo effects of certain complement inhibitors on systemic or local complement activation in a subject (e.g., a subject suffering from or at risk of a complement-mediated response) can also be assessed using in vitro assays such as those described herein or known in the art. Appropriate biological samples (e.g., plasma, synovial fluid, sputum) are obtained from the subject, e.g. prior to and following local administration of a complement inhibitor. The in vitro assay is performed using these samples as a source of complement components. Serial measurements beginning before administration of a complement inhibitor provide an indication of the extent to which the complement inhibitor inhibits complement activation and the time course and duration of the inhibition.

A number of different animal models with pathological features that resemble one or more features of a complement-mediated response are known in the art. A composition containing a complement inhibitor can be administered in various doses to mice, rats, dogs, primates, etc., that spontaneously exhibit a disorder or in which a disorder has been experimentally induced by subjecting the animal to a suitable protocol. The ability of the compound to prevent or treat one or more signs or symptoms of the disorder is assessed using standard methods and criteria.

Compounds that show promising results in animal studies, such as acceptable safety and feasibility of administering a dose expected to effectively inhibit complement in the relevant extravascular location in a human subject, may be tested in humans, e.g., using standard protocols and endpoints for clinical trials for therapies for the particular disorder under study.

### IV. Therapeutic Agents

The methods and compositions described herein encompass the use of a complement inhibitor in combination with a therapeutic agent. In the absence of the complement inhibitor, the therapeutic agent would otherwise adversely activate a complement pathway. Co-administration of a complement inhibitor with the complement-activating therapeutic may reduce or eliminate symptoms associated with a complement-mediated response, such as, for example, CARPA or CRS. Any therapeutic agent that is capable of complement activation, which can lead to complement-mediated responses such as CARPA or CRS, can be administered with a complement inhibitor as described herein to reduce, suppress or eliminate the deleterious effects of complement activation.

“Therapeutic agent” is used herein to refer to any pharmacologically active agent useful for treating a disorder. The term includes my pharmaceutically acceptable salt, prodrug, salt of a prodrug, and such derivatives of such an agent as are known in the art or readily produced using standard methods known in the art. “Prodrug” refers to a precursor of an agent, wherein the prodrug is not itself pharmacologically active (or has a lesser or different activity than the desired activity of the drug) but is converted, following administration (e.g., by metabolism) into the pharmaceutically active drug. A therapeutic agent is sometimes referred to as an “active agent” or “drug” herein. A therapeutic agent can be, without limitation, a small molecule or a biological macromolecule such as a polypeptide, antibody, or polynucleotide such as an aptamer, RNA agents such as interfering RNA (RNAi) agents or mRNA therapeutic agents, etc. The therapeutic effect of a polynucleotide can be mediated by the nucleic acid itself (e.g., antisense polynucleotide), or may follow transcription (e.g., RNAi, mRNA, interfering dsRNA, antisense RNA, ribozymes) or expression into a protein. The therapeutic effect of a protein (including an expressed protein) in treating a disorder can be accomplished by the protein remaining within a cell, remaining within the membrane of a cell, remaining attached to a cell membrane (intra- or extra-cellularly), remaining within the vicinity of an injection or delivery site, entering the bloodstream, and/or entering lymphatic system. Proteins include, but are not limited to, antibodies, hormones, cytokines, and growth factors. Small molecules include, but are not limited to, chemotherapeutic agents, anti-infective agents, inhibitors or agonists of intracellular target molecules, and vaccines.

In various embodiments, the therapeutic agent is a particle-encapsulated agent. By “particle-encapsulated agent” is meant a therapeutic agent that is contained within, e.g., a microparticle, a nanoparticle, a virus, or a liposome which is intended to protect (for example, from enzymatic degradation) the therapeutic agent during delivery of the agent to the intended target (such as a targeted tissue, cell or subcellular location) and/or to delay or sustain release of the therapeutic agent. The encapsulated therapeutic agent can be, in example, an encapsulated particle, an encapsulated micropartiele, an encapsulated nanoparticle, a encapsulated viral particle, or an encapsulated lipid, each of which is herein referred to as an encapsulated therapeutic.

In certain embodiments, the therapeutic agent, such as a polypeptide, antibody, polynucleotide, RNAi agent, mRNA therapeutic agent, the like, is encapsulated within a lipid nanoparticle. The term “lipid nanoparticle” or “LNP” refers to a particle of less than about 1,000 nm, typically less than about 200 nm, that is formulated with at least one lipid molecular species. Lipid nanoparticles include, but are not limited to, liposomes, irrespective of their lamellarity, shape, or structure. As used herein, a “liposome” is a structure having lipid-containing membranes enclosing us interior. Liposomes have one or more lipid membranes. Single-layered liposomes are referred to as “unilamellar,” and multi-layered liposomes are referred to as “multilamellar.” Lipid nanoparticles may further include one or more additional lipids and/or other components, which may be included in the liposome compositions for a variety of purposes, such as to stabilize a lipid membrane, to prevent lipid oxidation, or to attach ligands on the liposome surface. Any number of lipids may he present, including amphipathic, neutral, cationic, and anionic lipids. Lipid nanoparticles can be complexed with therapeutic agents, including polynucleotides, proteins, peptides, or small molecules and are useful as in vivo delivery vehicles.

In other embodiments, the therapeutic agent such as a poly peplide, antibody, polynucleotide, RNAi agent, mRNA therapeutic agent, or the like, is encapsulated in a viral particle, including but not limited to vital nanoparticles (“VNP”) and virus-like particles (“VLP”), each of which are useful for the sequestration and encapsulation of a therapeutic agent. The viral particle can be structured such that the internal cavity encapsulates the therapeutic agent and the external surface can optionally include targeting ligands to allow cell-specific delivery.

The viral particles may be formed from polypeptides derived from any virus known in the art and disclosed elsewhere herein. VLPs, for example, can be obtained from the nucleocapsid proteins of a virus selected from the group consisting of RNA-bacteriophages, adenovirus, papaya mosaic virus, influenza virus, norovirus, papillomavirus, hepadnaviridae, respiratory syncytial virus, hepatitis B virus, hepatitis C virus, measles virus; Sindbis virus; rotavirus, foot-and-mouth-disease virus, Newcastle disease virus, Norwalk virus, alphavirus; SARS, paramoxyvirus, transmissible gastroenteritis virus retrovirus, retrotransposon Ty, Polyoma virus; tobacco mosaic virus; Flock House Virus, Cowpea Chlorotic Mottle Virus; a Cowpea Mosaic Virus; and alfalfa mosaic virus.

“Treating”, as used herein, refers to providing treatment, i.e., providing, any type of medical or surgical management of a subject. The treatment can be provided to reverse, alleviate, inhibit the progression of, prevent or reduce the likelihood of a disorder or condition, or to reverse, alleviate, inhibit or prevent the progression of, prevent or reduce the likelihood of one or more symptoms or manifestations of a disorder or condition. “Prevent” refers to causing a disorder or condition, or symptom or manifestation of such not to occur for at least a period of time in at least some individuals. Treating can include administering an agent to the subject following the development of one or more symptoms or manifestations indicative of a complement-mediated condition such as CARPA or CRS, e.g., to reverse, alleviate, reduce the severity of and/or inhibit or prevent the progression of the condition and/or to reverse, alleviate, reduce the severity of, and/or inhibit or one or more symptoms or manifestations of the condition. According to the methods described herein, a composition can be administered to a subject who has developed a complement-mediated response or is at increased risk of developing such a disorder relative to a member of the general population. Such a composition can be administered prophylactically, i.e., before development of any symptom or manifestation of the condition. Typically in this case the subject will be at risk of developing the condition, for example, when exposed to a complement-activating composition, e.g., a particle or nanoparticle encapsulated therapeutic, e.g., a viral particle used in gene therapies or a therapeutic agent delivered by, for example, a lipid nanoparticle.

In various embodiments, the therapeutic agent and the complement inhibitor are administered concurrently. “Concurrent administration” as used herein with respect to two or more agents, e.g., therapeutic. agents, is administration performed using doses and time intervals such that the administered agents are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks, etc. Accordingly, the agents may, but need not be, administered together as part of a single composition. In addition, the agents may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). Agents administered within such time intervals may be considered to be administered at substantially the same time. In certain embodiments, concurrently administered agents are present at effective concentrations within the body (e.g., in the blood and/or at a site of local complement activation) over the time interval. When administered concurrently, the effective concentration of each of the agents needed to elicit a particular biological response may be less than the effective concentration of each agent when administered alone, thereby allowing a redaction ose of one or more of the agents relative to the dose that would be needed if the agent was administered as a single agent. The effects of multiple agents may, but need not be, additive or synergistic. The agents may be administered multiple times. The non-negligible concentration of an agent may be, for example, less than approximately 5% of the concentration that would be required to elicit a particular biological response, e.g., a desired biological response.

In certain embodiments, the complement inhibitor is conjugated to the thertherapeutic agent, or conjugated to the delivery system for the therapeutic agent. In other embodiments, the complement inhibitor is conjugated to the delivery system, such as the encapsulated particle, e.g., the encapsulated nanoparticle or viral particle. Suitable methods for conjugating heterologous moieties, such as a therapeutic agent, e.g., a polypeptide, an antibody, a polynucleotide, an RNAi agent, an RNA therapeutic agent, and the like, and/or the delivery system to a complement inhibitor are known in the art. A stable linkage between the conjugated moieties (e.g., the therapeutic agent and the complement inhibitor) can be obtained using a non-cleavable or a cleavable linker. Non-limiting examples of linkers include, but are not limited to, amide, carbamtate, carbonate, lactone, lactam, carboxylate, ester, cycloalkene, cyclohexene, heteroalicyclic heteroaryl, triazine, triazole, disulfide, imine, imide, oxime, aldiminie, ketimine, hydrazone, semicarbazone, acetal, ketal, aminal, aminoacetal, thioacetal, thioketal, phosphate ester, and the like. Viral coat proteins can also be chemically modified using bioconjugation protocols. Amino acids with reactive side chains such lysine, cysteine, aspartate and glutamate can be functionalized with antibodies, polynucleotides, peptides, and the like, using, for example, N-hydroxysuccinimidyl ester (NHS), maleimide, isothiocyanate and carbodiimide chemistries.

An “effective amount” of an active agent such as a therapeutic agent or a complement inhibitor refers to the amount of the active agent sufficient to elicit a desired biological response (or, equivalently, to inhibit an undesired biological response). The absolute amount of a particular agent that is effective may vary depending an such factors as the desired biological endpoint, the agent to be delivered, the target tissue, etc. An “effective amount” may be administered in a single dose, or may be achieved by administration of multiple doses. An effective amount of the therapeutic agent, for example, may be an amount sufficient to relieve at least one symptom of a disorder. An effective amount may be an amount sufficient to slow the progression of a chronic and progressive disorder, e.g., to increase the time before one or more symptoms or signs of the disorder manifests itself or to increase the time before the individual suffering from the disorder reaches a certain level of impairment. An effective amount may be an amount sufficient to allow faster or greater recovery from an injury than would occur in the absence of the agent. An effective amount of a co-administered or conjugated complement inhibitor would be, for example, an amount sufficient to at least locally and temporarily reduce, suppress or eliminate adverse effects of complement activation, e.g., CRS or CARPA, caused by administration of the therapeutic agent.

A. mRNA Therapy

In some embodiments, the therapeutic agent is an mRNA treatment, especially wherein the therapeutic agent is delivered by a particle delivery vehicle, e.g., a nanoparticle, e.g., a lipid nanoparticle. Thus, the compositions and methods described herein provide for the administration of a therapeutic mRNA in combination with a complement inhibitor. In particular, the compositions and methods described herein are suitable for the treatment of diseases or disorders relating to the deficiency of proteins and/or enzymes that are excreted or secreted by the target cell into the surrounding extracellular fluid (e.g., mRNA encoding hormones and neurotransmitters). In some embodiments, the therapeutic mRNA is a vaccine. In some embodiments, the mRNA therapeutic agent is useful for treating, for example, Crigler-Najjar syndrome, primary hyperoxaluria type 1 (PHI), various acidemias (including, for example, proprionic acidemia, argininosuccinic aciduria and methylmalonic acidemia), myocardial ischemia, Huntington's Disease; Parkinson's Disease; muscular dystrophies (such as, e.g. Duchenne and Becker); hemophilia diseases (such as, e.g., hemophilia B (FIX), hemophilia A (FVIII); SMN1-related spinal muscular atrophy (SMA); amyotrophic lateral sclerosis (ALS); GALT-related galactosemia; Cystic Fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR1-related disorders, which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type Cl; the neuronal ceroid lipofuscinoses-related diseases including Juvenile Neuronal Ceroid Lipofuscinosis (JNCL), Juvenile Batten disease, Santavuori-Halta disease, Jansky-Bielschowsky disease, and PTT-1 and TPP1 deficiencies; argininosuccinate synthetase deficiency; EIF2B1, EIF2B2, EIF2B3, EIF2B4 and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy's disease (SBMA); thrombotic thromboeytopenic purpura (TTP); ornithine transcarbamylase deficiency (OTCD); Leber's hereditary optic neuropathy (LHON); phenylketonuria (PKU), glycogen storage disorders (GSDs) including, for example, GSD1a; Notch-3 related cerebral autosomal dominant arteriopathy with subcortical infarcts and leukoencephalopathy (CADASIL); SCN1A and SCN1B-related seizure disorders; the Polymerase G-related disorders, which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions, X-Linked adrenal hypoplasia; X-linked agammaglobulinemia; Wilson's disease; and Fabry Disease, In one embodiment, the nucleic acids, and M particular mRNA, of the invention may encode functional proteins or enzymes that are secreted into extracellular space. For example, the secreted proteins include clotting factors, components of the complement pathway, cytokines, chemokines, chemoattractrmts, protein hormones (e.g. EGF, PDF), protein components of serum, antibodies, secretable toll-like receptors, and others. In some embodiments, the compositions of the present invention may include mRNA encoding erythropoietin, α1-antitrypsin, carboxypeptidase N or human growth hormone.

Where mRNA therapeutics are delivered as a particle e.g., nanoparticle, e.g., a lipid nanoparticle, encapsulated therapeutic, there is a significant likelihood that adverse complement-mediated activation will occur. As used herein, the phrase “lipid nanoparticle” or “LNP” refers to an encapsulation vehicle comprising one or more lipids (e.g., cationic lipids, non-cationic lipids, and PEG-modified lipids). LNPs can he formulated to deliver one or more mRNA to one or more target cells. Examples of suitable lipids include, tbr example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoaciylates, polylactide, polylactide-polyglycolide copolymers, polyeaprolactones, dextran, albumin, gelatin, alginate, collagen, chnosan, cyclodextrins, dendrimets and polyethylenimine. In one embodiment, the transfer vehicle is selected based upon its ability to facilitate the transfection of a mRNA to a target cell.

### B. Antibody Therapy

In another embodiment, the therapeutic agent is an antibody. As used throughout the present disclosure, the term “antibody” refers to a whole or intact antibody (e.g., IgM, IgG, IgA, IgD, or IgE) molecule that is generated by any one of a variety of methods that are known in the art and described herein. The term “antibody” includes a polyclonal antibody, a monoclonal antibody, a chimerized or chimeric antibody, a humanized antibody, a deimmumzed human antibody, and a fully human antibody. The antibody can be made in or derived from any of a variety of species, e.g., mammals such as humans, non-human primates (e.g., monkeys, baboons, or chimpanzees), horses, cattle, pigs, sheep, goats, dogs, cats, rabbits, guinea pigs, gerbils, hamsters, rats, and mice. The antibody can be a purified or a recombinant antibody.

### C. Gene Therapy

In another, embodiments, the therapeutic agent is a gene therapy, in this embodiment, nucleic acids encoding a therapeutic peptide or RNA molecule can be incorporated into a viral vector construct to be used as a part of a gene therapy protocol to deliver nucleic acids that can be used to express and produce agents within cells. Expression constructs of such components can be administered in any therapeutically effective carrier, e.g., any formulation or composition capable of effectively delivering the component gene to cells in vivo or ex viva. Approaches include providing the subject nucleic acid in viral vector(s) including, for example, recombinant retroviruses, adenovirus, adeno-associated virus, lentivirus, herpes simplex virus-1, (HSV-1), or recombinant bacterial or eukaryotic plasmids. Viral vectors can transfect cells directly; plasmid DNA can be delivered with the help of, for example, cationic liposomes (lipofectin) or derivatized (e.g., antibody conjugated), polylysine conjugates, gramicidin S, artificial viral envelopes or other such intracellular carriers, as well as direct injection of the gene construct or CaPO₄ precipitation (see, e.g., WO04/060407) carried out in vivo. Examples of suitable retroviruses include pLJ, pZIP, pWE and pEM (Eglitis, M. et al., Science, 230:1395-8, 1985; Danos, O. & Mulligan, R., Proc. Natl. Acad. Sci. USA, 85:6460-4, 1988; Wilson, J. et al, Proc. Natl. Acad. Sci. USA, 85:3014-8, 1988; Armentano, D. et al., Proc. Natl. Acad. Sci. USA, 87:6141-5, 1990; Huber, B. et al., Proc. Natl. Acad. Sci. USA, 88:8039-43, 1991; Ferry, N. et al., Proc. Natl. Acad. Sci. USA, 88:8377-81, 1991; Chowdhury, J. et al., Science, 254:1802-5, 1991; van Beusechem, V. et al., Proc. Natl. Acad. Sci. USA, 89;7640-4, 1992; Kay, M. et al., Human Gene Ther., 3:641-7, 1992; Dai, Y. et al., Proc. Natl. Acad. Sci. USA, 89;10892-5, 1992; Hwu, P. et al., J. Immunol., 150:4104-15, 1993; U.S. Pat. Nos. 4,868,116 and 4,980,286: PCT Publication Nos. WO89/07136, WO89/02468, WO89/05345, and WO92/07573). Another viral gene delivery system utilizes adenovirus-derived vectors (Berkner, K., BioTechniques, 6:616-29, 1988; Rosenfeld, M et al., Science, 252:431-4, 1991; Rosenfeld, M. et al., Cell, 68:143-55, 1992). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno associated virus (AAV)(Flotte, T. et al., Am. J. Respir. Cell Mol. Biol., 7:349-56, 1992; Samidski, R. et al., J. Virol., 63:3822-8, 1989; McLaughlin, S. et al., J. Virol., 62:1963-73, 1988).

### D. Additional Therapeutic Agents

Described herein are therapeutic methods combining the use of complement inhibitors with one or more therapeutic agents effective for treatment of a disorder disclosed herein. The therapeutic agent can include, for example, anti-inflammatory agents such as corticosteroids, non-steroidal anti-inflammatory agents, leukotriene or leukotriene receptor antagonists, cytokine or cytokine receptor antagonists (e.g., anti-TNF-alpha agents such as antibodies or soluble TNF-alpha receptors or fragments thereof that bind TNF-alpha), anti-IgE agents (e.g. antibodies or antibody fragments that bind to IgE or to an IgE receptor), angiogenesis inhibitors, analgesic agents, and anti-infective agents. Anti-infective agents include anti-viral agents, anti-bacterial agents, anti-fungal agents, and anti-parasite agents. Suitable corticosteroids agents of use in various embodiments of the invention include dexamethasone, cortisone, prednisone, hydrocortisone, beclomethasone dipropionate, betamethasone, flunisolide, methylprednisone, paramethasone, prednisolone, triamcinolone, alclometasone, amcinonide, clobetasol, fludrocortisone, diflorasone diacetate, fluocinolone acetonide, fluocitamide, fluorometholone, flurandrenolide, halcinonide, medrysone and mometasone, and pharmaceutically acceptable mixtures and salts thereof and any other derivatives and analogs thereof. Antibiotics such as sulfisoxazoie, penicillin G, ampicillin, cephalosporins, quinolones, amikacin, gentamicin, tetracyclines, chloramphenicol, erythromycin, clindamyoin, isoniazid, rifampin, and derivatives, salts and mixtures thereof; antifungals such as amphotericin B, nystatin, ketoconazole, itraconazole; and other art known anti-infective or agents or combinations thereof are of use.

### V. Methods for Administration of Treatment

The above-described compositions are useful in, inter alia, methods for treating or preventing a variety of complement-associated disorders in a subject, e.g., CARPA or CRS, that arise in conjunction with, or due to administration of a therapeutic agent that activates a complement pathway. The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject (U.S. Patent Application Publication No. 20080241223; U.S. Pat, Nos. 5,501,856; 4,863,457; and 3,710,795; EP488401; and EP 430539, the disclosures of each of which are incorporated herein by reference in their entirety). The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, a therapeutic agent is delivered to a subject by way of local administration. As used herein, “local administration” or “local delivery,” refers to delivery that does not rely upon transport of the composition or agent to its intended target tissue or site via the vascular system. The composition can be delivered, for example, by injection or implantation of the composition or agent or by injection or implantation of a device containing the composition or agent. Following local administration in the vicinity of a target tissue or site, the composition or agent, or one or more components thereof, may diffuse to the intended target tissue or site.

The present disclosure also presents controlled-release or extended-release formulations of therapeutic agents that are suitable for chronic and/or self-administration of the agent. The various formulations can be administered to a patient in need of treatment with the medication as a bolus or by continuous infusion over a period of time.

In some aspects, the delivery agent comprises a lipidoid, a liposome, lipoplex, a LNP, a polymeric compound, a peptide, a protein, a cell, a nanopartiele mimic, a nanotube, or a conjugate. In some aspects, the delivery agent is a LNP. In some aspects, the LNP comprises the lipid selected from the group consisting of DLin-DMA, DLin-K-DMA, 98N12-5, C12-200, DLin-MC3-DMA, DLin-KC2-DMA, DODMA, PLGA, PEG, PEG-DMG, PEGylated lipids, amino alcohol lipids, KL22, and combinations thereof. In some aspects, the therapeutic agent and/or the complement inhibitor are formulated for subcutaneous, intravenous, intraperitoneal, intramuscular, intra-articular, intra-synovial, intrasternal, intrathecal , intrahepatic, intralesional intracranial, intraventricular, oral, inhalation spray, topical, rectal, nasal, buccal, vaginal, intratumoral, or intradermal in vivo delivery.

### VI. Pharmaceutical Compositions

Compositions containing a complement inhibitor described herein can be formulated as a pharmaceutical composition, e.g., for administration to a subject for the treatment or prevention of a complement-associated response. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (Berge, S. et al., J. Pharm. Sci., 66;1-19, 1977).

The compositions can be formulated according to .standard methods. Pharmaceutical formulation is a well-established art, and is further described in, e.g., Gennaro (2000) “Remington: The Science and Practice of Pharmacy,” 20th Edition, Lippincott, Williams & Wilkins (ISBN: 0683306472); Ansel et al. (1999) “Pharmaceutical Dosage Forms and Drug Delivery Systems,” 7th Edition, Lippincott Williams & Wilkins Publishers (ISBN: 0683305727); and Kibbe (2000) “Handbook of Pharmaceutical Excipients American Pharmaceutical Association,” 3rd Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 11/2 years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing an antibody or fragment intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular intramuscular, intraarterial, intrathecal, intracapsular, intraorbital, intracardiac, intradermal, intraperitoneal, transtracheal, subcutaneous, subcuticular, intraarticuiar, subcapsular, subarachnoid, intraspinal, epidural, intracerebral, intracranial, intracarotid and intrasternal injection and infusion.

The compositions can be formulated as a solution, microemulsion, dispersion, liposome, or other ordered structure suitable for stable storage at high concentration. Sterile injectable solutions can be prepared by incorporating an antibody (or a fragment of the antibody) described herein in the required amount in an appropriate solvent with one or a combination of ingredients enumerated above, as required, followed by filtered sterilization. Dispersions are generally prepared by incorporating an antibody or fragment described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of an antibody, or an antigen-binding fragment thereof, described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be bmught about by including in the composition a reagent that delays absorption, for example, monostearate salts, and gelatin.

The complement inhibitor described herein can also be formulated in immunoliposome compositions. Liposomes containing the inhibitor can be prepared by methods known in the art (Eppstein, E. et al., Proc. Natl. Acad. Sci. USA, 82:3688-92, 1985; Hwang, K. et al., Proc. Natl. Acad. Sci. USA, 77:4030-4, 1980; U.S. Pat. Nos. 4,485,045 and 4,544,545). Liposomes with enhanced circulation time are disclosed in, e.g., U.S. Pat. No. 5,013,556.

In certain embodiments, the complement inhibitor can be prepared with a carrier that protects the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and potylactic acid (J. R. Robinson (1978) “Sustained and Controlled Release Drug Delivery Systems,” Marcel Dekker, Inc., New York).

In some embodiments, the complement inhibitor described herein can be formulated with one or more additional active agents useful for treating or preventing a complement-associated disorder in a subject. Additional agents for treating a complement-associated disorder in a subject vary depending on the particular disorder being treated, but can include, without limitation, an antihypertensive (e.g., an angiotensin-converting enzyme inhibitor), an anticoagulant, a corticosteroid (e.g., prednisone), or an immunosuppressive agent (e.g., vincristine or cyclosporine A). Examples of anticoagulants include, e.g., warfarin (Coumadin), heparin, phenindione, fondaparinux, idraparinux, and thrombin inhibitors (e.g., argatroban, lepirudin, bivalitudin, or dabigatran). An antibody or fragment thereof described herein can also be formulated with a fibrinolytic agent (e.g., ancrod, ε-aminocaproic acid, antiplasmin-a₁, prostacyclin, and defibrotide) for the treatment of a complement-mediated response. In some embodiments, the complement inhibitor can be formulated with a lipid-lowering agent such as an inhibitor of hydroxymethylglutaryl CoA reductase. In some embodiments, the complement inhibitor can be formulated with, or for use with, an anti-CD20 agent such as rituximab (RITUXAN®; Biogen Idec, Cambridge, Mass.). In some embodiments, e,g., for the treatment of RA, the the complement inhibitor can be formulated with one or both of infliximab (REMICADE®; Centocor, Inc.) and methotrexate (RHEUMATREX®, TREXALL®). In some embodiments, the complement inhibitor described herein can be formulated with a non-steroidal anti-inflammatory drug (NSAID). Many different NSAIDS are available, some over the counter including ibuprofen (ADVIL®, MOTRIN®, NUPRIN®) and naproxen (ALLEVE®) and many others are available by prescription including meloxicam (MOBIC®), etodolac (LODINE®), nahumetone (RELAFEN®), sulindac (CLINORIL®), tolementin (TOLECTIN®), choline magnesium salicylate (TRILASATE®), diclofenac (CATAFLAM®, VOLTAREN®, ARTHROTEC®), diflusinal (DOLOBID®), indomethicin (INDOCIN®), ketoprofen (ORUDIS®, ORUVAIL®), oxaprozin (DAYPRO®), and piroxicam (FELDENE®). In some embodiments the complement inhibitor can be formulated for use with an anti-hypertensive, an anti-seizure agent (e.g., magnesium sulfite), or an anti-thrombotic agent. Anti-hypertensives include, e.g., labetalol, hydralazine, nifedipine, calcium channel antagonists, nitroglycerin, or sodium nitroprussiate (Mihu, D. et al., J. Gasrointestin, Liver Dis., 16:419-24, 2007). Anti-thrombotic agents include, e.g., heparin, antithrombin, prostacyclin, or low dose aspirin.

### EXAMPLES

Terminal inhibition of complement dramatically reduces the cytokine storm (i.e., cytokine release syndrome or CRS) associated with each injection of formulated mRNA in lipid nanoparticles (LNPs). The cytokine storm can potentially boost the adaptive immune response and induce an immune reaction to the LNP-formulated mRNA or other gene therapy product over time. While not bound to any particular theory or mechanism, this reaction may contribute to a reduction in efficacy of the mRNA therapy over time.

A short-acting complement inhibitor, for example a short-acting C5 inhibitor or factor H, can inhibit terminal complement activity for about 20 minutes to an hour. Administration of these inhibitors was demonstrated to be safe in more than one thousand patients. In an embodiment, a short-acting C5 inhibitor is used together with lipid nanoparticles or other delivery formulations to reduce the associated cytokine storm and allowing for the reduction of immunogenicity to, for example, particle-encapsulated (e.g., nanoparticle-encapsulated) therapeutics, including, for example, mRNA and siRNA; and/or gene therapy agents. A short-acting complement inhibitor can be used repeatedly, without marked impact on innate immune responses or safety.

### Example 1

A single dose of 0.5 mg/kg in PBS (buffer control), luciferase mRNA, human erythropoietin (hEPO) mRNA, and hEPO protein was administered to 8 to 10-week-old male balb/cJ mice and immune response was evaluated. As shown in FIG. 1, a single dose of mRNA administration elicited a cytokine response (IL-6, KC/GRO and TNF-alpha) at 2 and 6 hours, with the response returning to baseline by 24 hours.

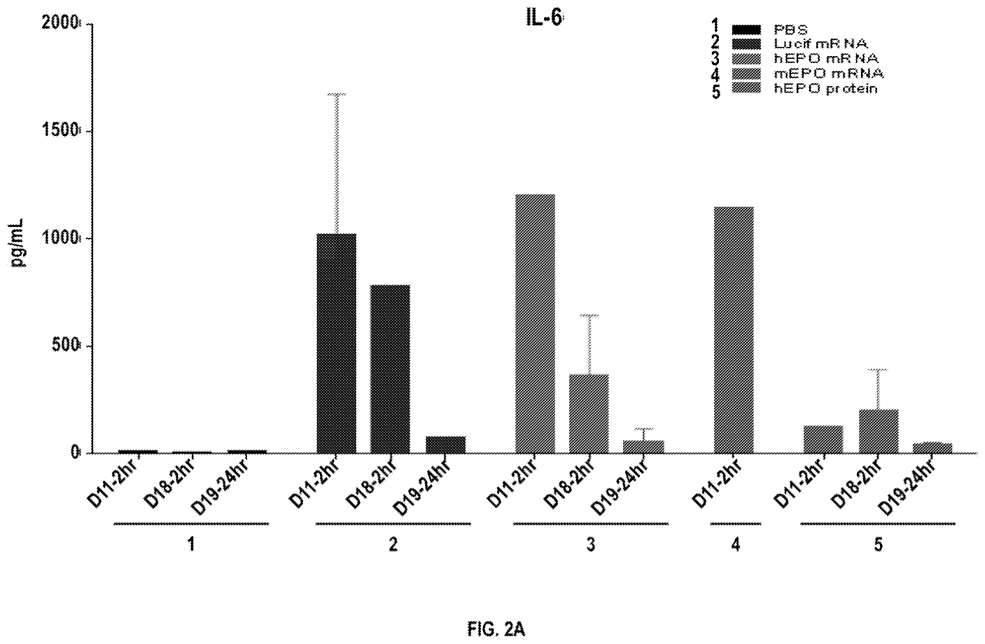

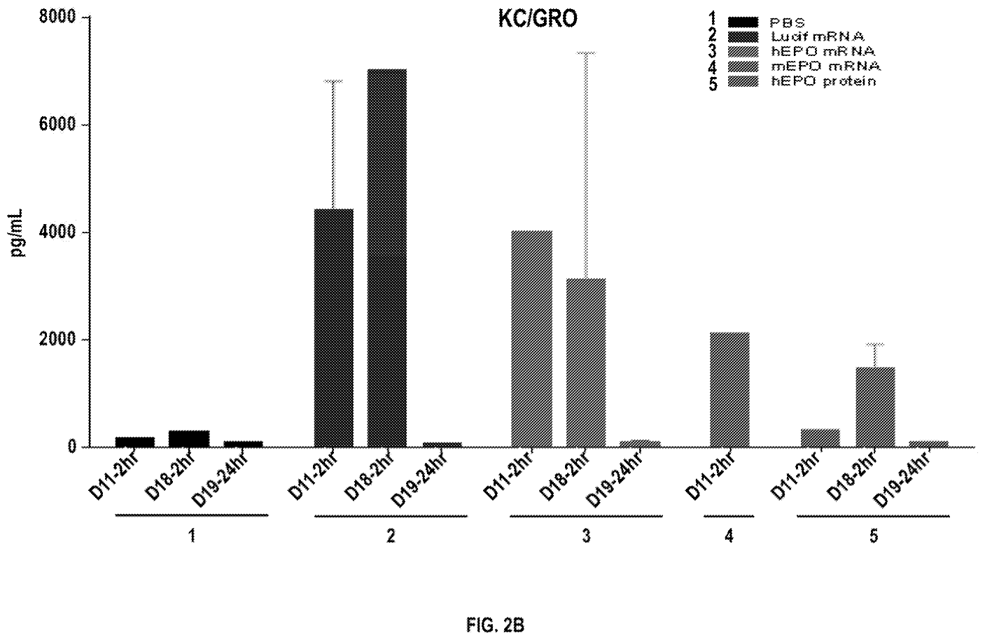

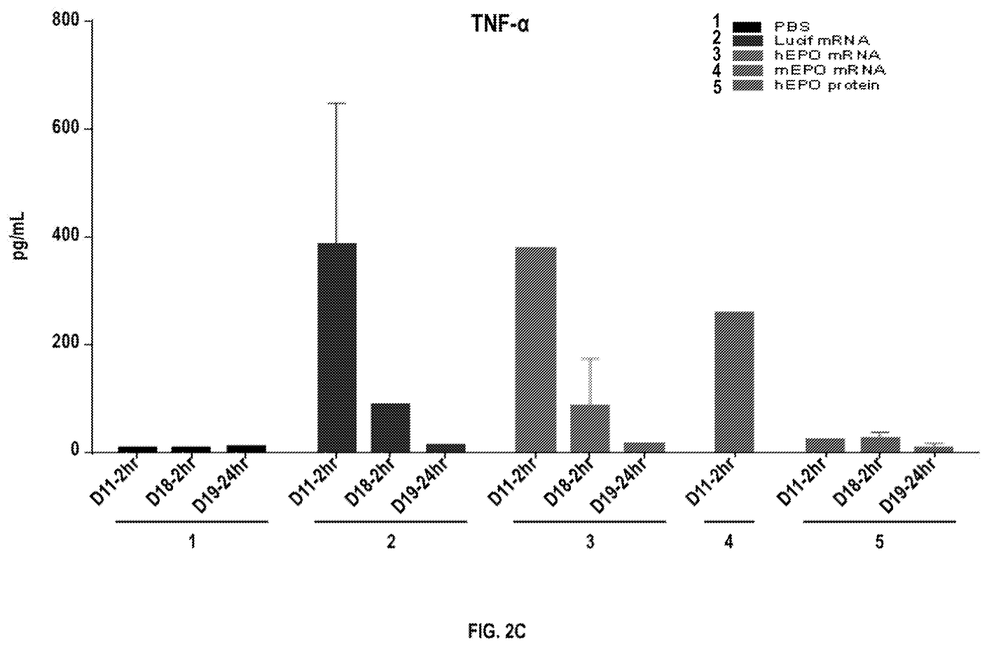

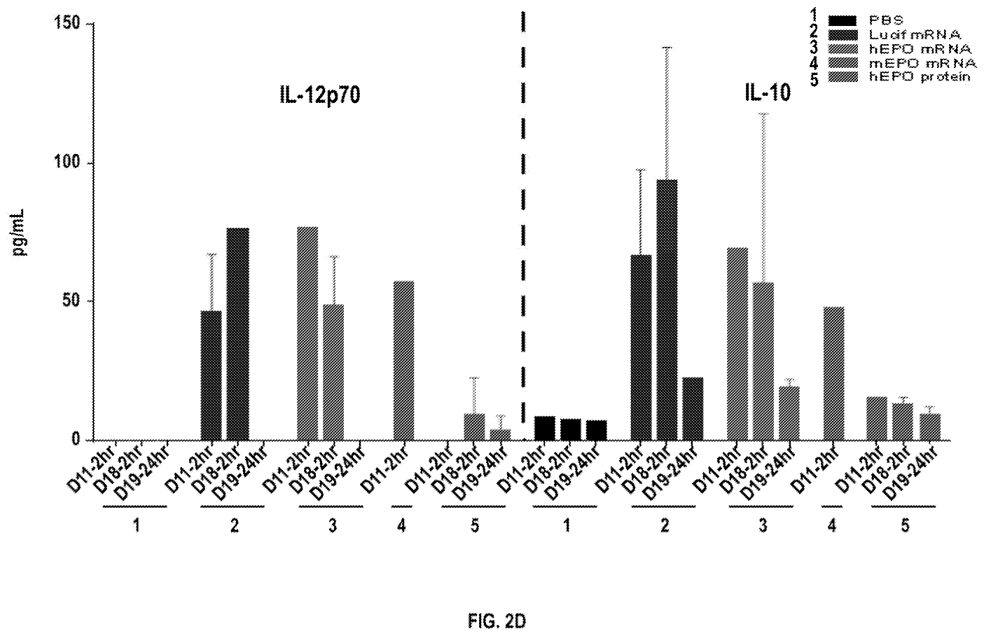

In further experiments, a single dose a single dose containing LNP formulated hEPO mRNA and murine EPO (mEPO) mRNA, and further containing hEPO protein was administered to 12 to 14-week-old male BALB/c mice and immune response was evaluated. The mRNA was formulated using Lipid enabled and Unlocked Nucleic Acid modified RNA (LUNAR™). FIG. 2 shows that the LNP mRNA elicited dose dependent cytokine responses at 2 and 6 hours for 1L-6, KC/GRO, TNF-alpha, and IL-12, which was resolved by 24 hours.

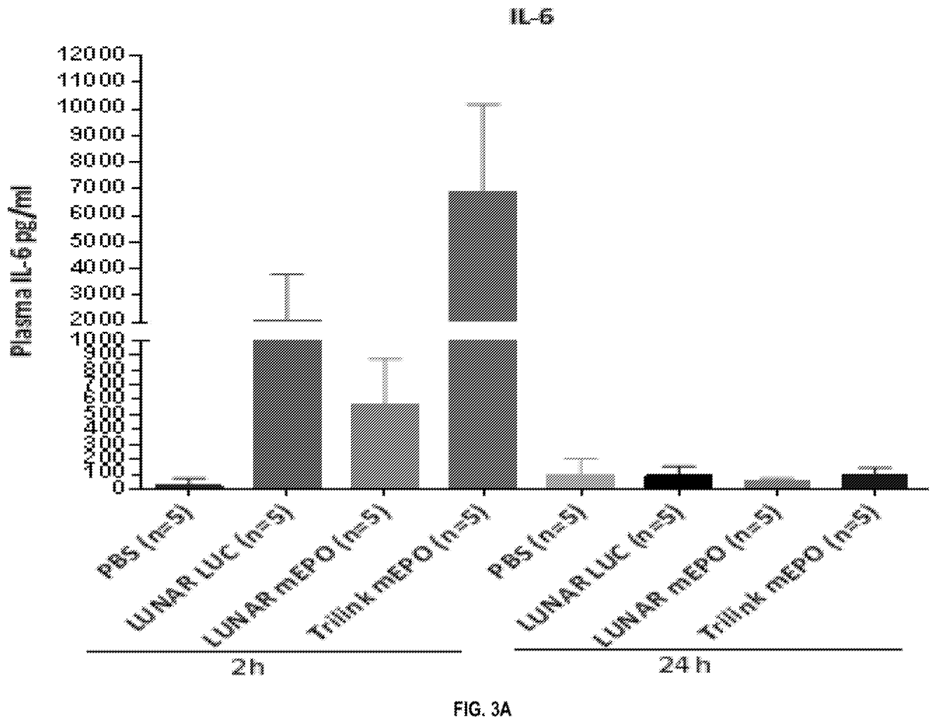

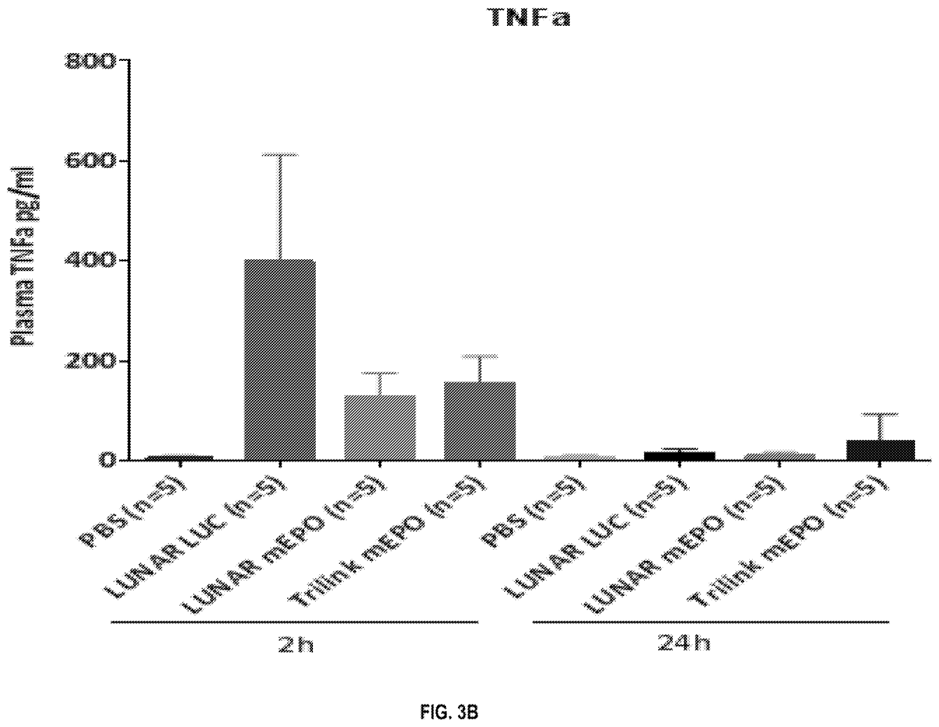

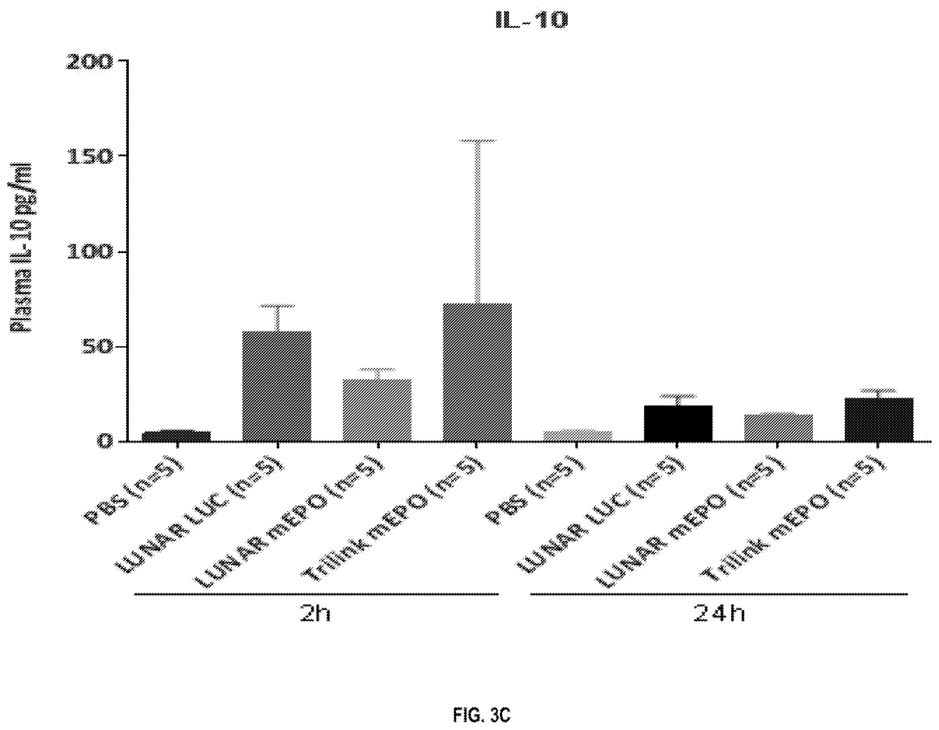

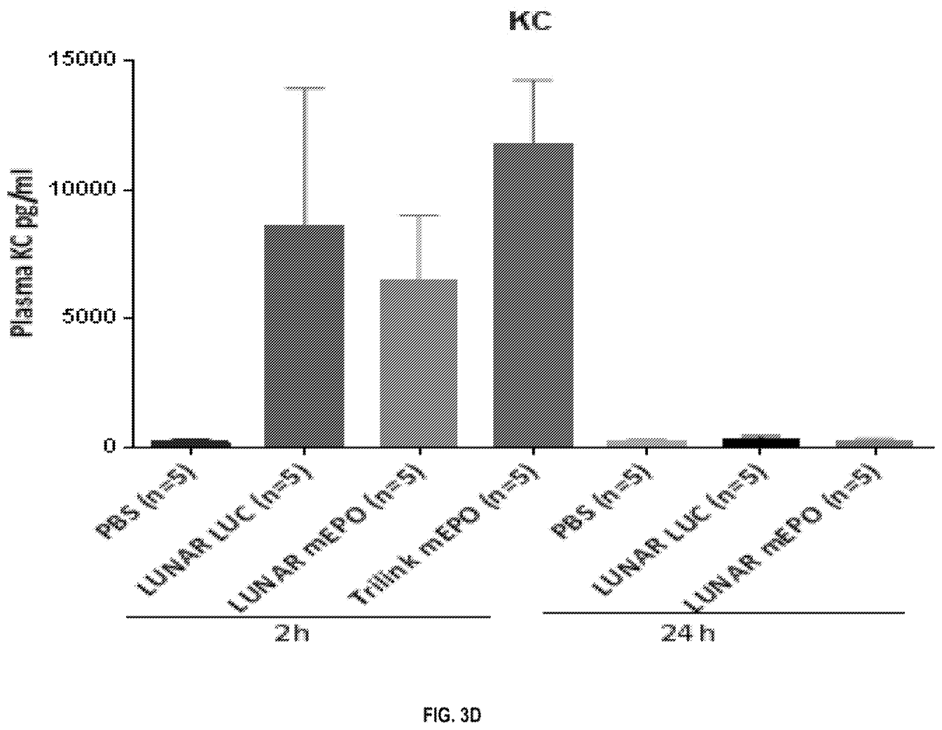

The LNP mEPO mRNA formulated as LUNAR™ or formulated by TriLink in S9K were further tested in weekly serial administrations of 0.5 mg/kg to 9-week-old male Balb/cJ mice. After 6 weeks, plasma IL-6, TNF-alpha, IL-10 and KC were elevated at 2 hours and resolved by 24 hours (FIG. 3).

### Example 2

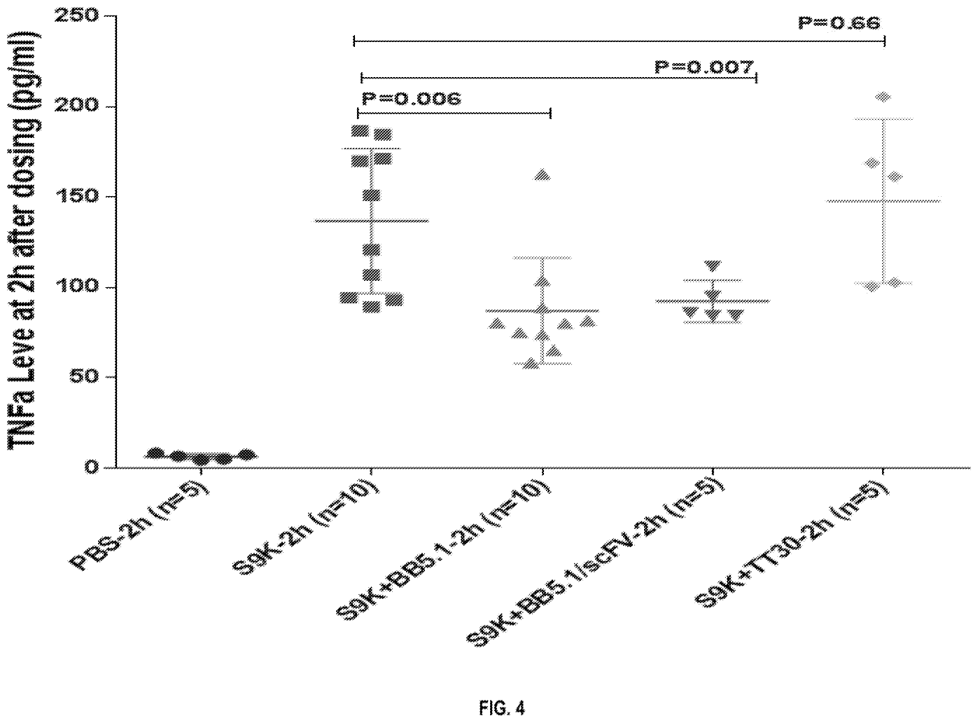

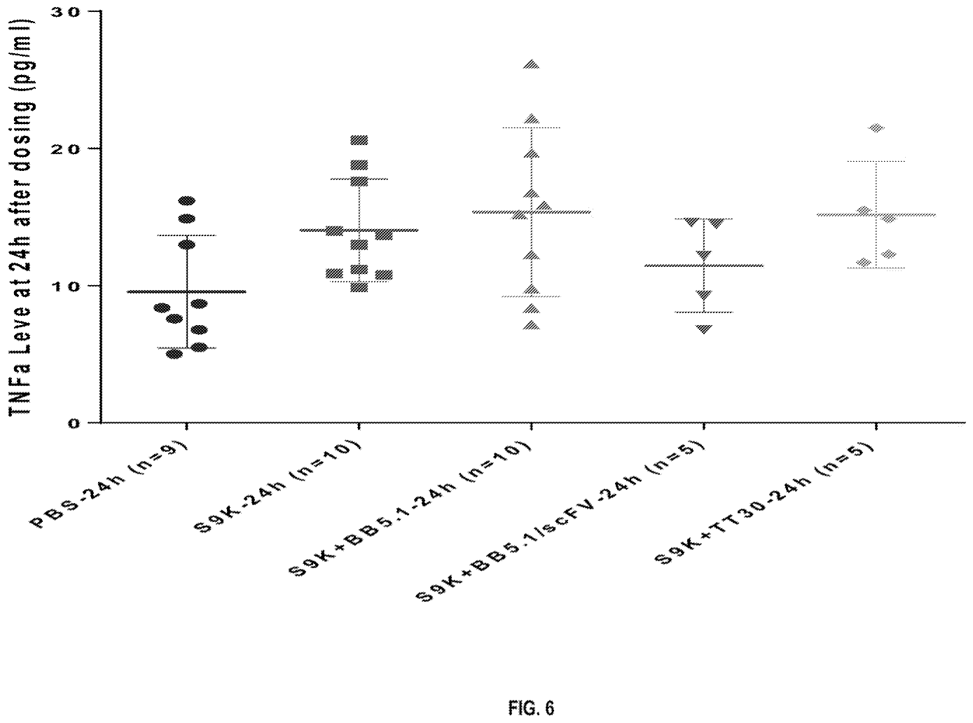

To evaluate the induction of cytokine response and validate the action of a short-acting complement inhibitor with a single intravenous (IV) dose, male Balb/cJ mice (12-14 weeks old) were injected with PBS, 0.5 mg/kg S9K LNPs formulated with TriLink mEPO mRNA (S9K), S9K+40 mg/kg BB5.1, S9K+10 mg/kg BB5.1 scFV, or S9K+40 mg/kg mTT30. Plasma inflammatory cytokines were measured at specified times (see, e.g., FIGS. 4-6, and Table 1 below).

TABLE 1

| | | | Measurement |
|---|---|---|---|
| Group (n = 5) | Route | Dose (single) | Times (hours) |
| PBS | IV | N/A | 2, 6, 24 |
| S9K | IV | 0.5 mg/kg | 2, 6, 24 |
| S9K + BB5.1 | IV | 0.5 mg/kg + 40 mg/kg | 2, 6, 24 |
| S9K + BB5.1 scFV | IV | 0.5 mg/kg + 10 mg/kg | 2, 6, 24 |
| S9K + mTT30 | IV | 0.5 mg/kg + 40 mg/kg | 2, 6, 24 |

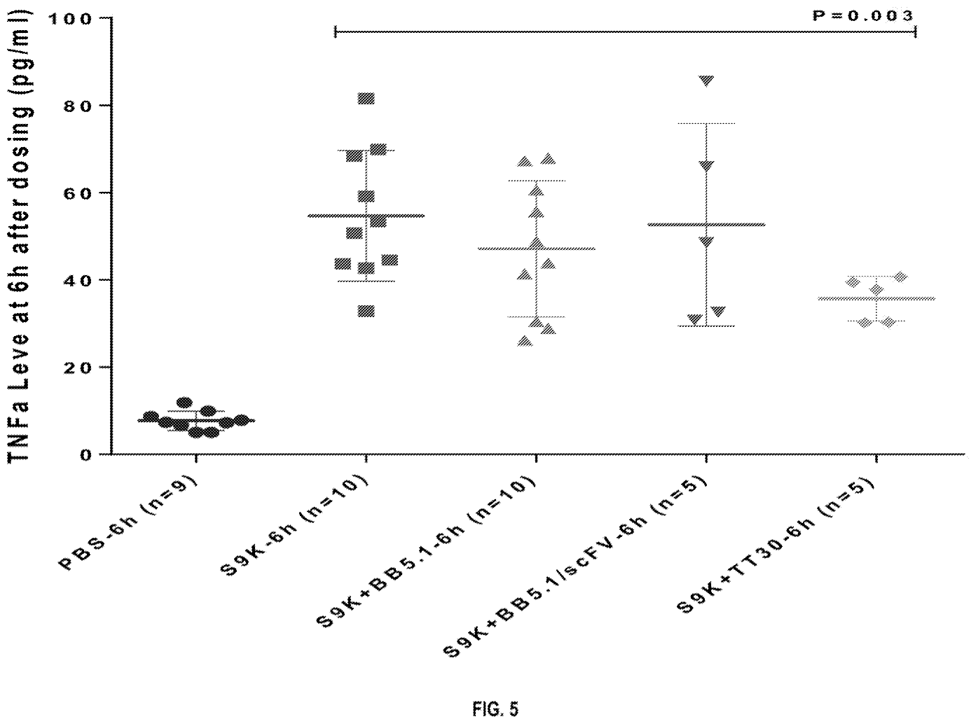

Administration of long and short acting C5 inhibitors (BB5.1 and BB5.1 scFV, respectively) resulted in a reduction of levels of TNF-alpha at two hours post injection of formulated mRNA (FIG. 4), with the short-acting C5 inhibitor TT30 showing a higher reduction in TNF-alpha at 6 hours compared to two hours (FIG. 5). The effectiveness of TT30, or any inhibitor of the alternative complement pathway may vary depending on the size, chemistry and molecular composition of a particular delivery formulation. These results show that inhibitors of the terminal components of the complement pathway, regardless of the half-life, are effective in blocking cytokine release associated with injection of a therapeutic agent in an LNP formulation.

What is claimed is:

1. A method tbr reducing or eliminating a complement-mediated response in a patient receiving treatment for a disease or disorder, wherein the treatment comprises one or more therapeutic agents that induce or are likely to induce a local or systemic complement-mediated response, comprising administering one or more complement inhibitors to the patient.

2. The method of claim 1, herein the complement-mediated response is Complement Activation-Related Pseudoallergy (CARPA) or Cytokine Release Syndrome (CRS).

3. The method of claim 2, wherein the complement-mediated response is CARPA.

4. The method of claim 2, wherein complement-mediated response is CRS.

5. The method of claim 1, wherein the treatment comprises a gene therapy agent, an mRNA therapeutic, an antibody therapeutic, or a cell therapy agent.

6. The method of claim 1, wherein the one or more therapeutic agent(s) are administered to the patient with a lipid-based drug delivery system.

7. The method of claim 6, wherein the one or more therapeutic agent(s) are encapsulated within or conjugated to a lipid nanoparticle, at nanostructured lipid carrier, a lipid drug conjugate-nanoparticle, a liposome, at transfersome, an ethosome, a liposphere, a niosome, a cubosome, a virosome, an iscom, a nanoemulsion, or a phytosome.

8. The method of any one of claims 1-7, wherein the one or more complement inhibitors inhibits an enzymatic activity of a soluble complement protein in the patient.

9. The method of any one of claims 1-7, wherein the one or more complement inhibitors inhibits cleavage of a complement component selected from the group consisting of: C5, C6, C7, C8, C9, factor D and factor B.

10. The method of any one of claims 1-7, wherein the one or more complement inhibitors inhibits cleavage of C5.

11. The method of any one of claims 1-7, wherein the one or more complement inhibitors is a peptide, a fusion protein, an antibody, a small molecule or an aptamer.

12. The method of any of claims 1-7, wherein in the one or more therapeutic agent(s) and the complement inhibitor are administered concurrently.

13. The method of any one of claims 1-7, wherein the one or more complement inhibitors are administered locally.

14. method of claim 13, wherein the one or more complement inhibitors is administered at an extravascular location.

15. The method of claim 13, wherein the one or more therapeutic agents is administered by an administration method selected from the group consisting of subcutaneous, intraperitoneal, intramuscular, intra-articular, intra-synovial, intrasternal, intrathecal, intrahepatic, intralesional, intracranial, intraventricular, oral, pulmonary, topical, rectal, nasal, buccal, vaginal, intratumoral and intradermal.

16. The method of claim 14, wherein the one or more complement inhibitors is a peptide or an antibody that binds to a soluble complement protein that is produced at the extravascular location.

17. The method of any one of claims 1-7, wherein the one or more complement inhibitors is administered in an amount sufficient to produce a clinically significant reduction in severity of at least one symptom of CARPA or CRS, as compared to when the one or more complement inhibitors is not administered with the one or more therapeutic agents.

18. The method of claim 17, wherein the clinically significant reduction in severity of at least one symptom of CARPA or CRS, as compared to when the one or more complement inhibitors is not administered with the one or more therapeutic agents, is resolved after a period of about 4 hours following administration of the one or more complement inhibitors.

19. A pharmaceutical composition comprising:
a. a composition comprising one or more therapeutic agents, wherein the composition induces or is likely to induce a local or systemic complement-mediated response; and
b. one or more complement inhibitors capable of inhibiting a complement-mediated response.

20. The pharmaceutical composition of claim 19, wherein the one or more therapeutic agents include a gene therapy agent, an mRNA therapeutic, an antibody therapeutic, or a cell therapy agent.

21. The pharmaceutical composition of claim 19, wherein the one or more therapeutic agents is formulated in a lipid drug delivery system.

22. The pharmaceutical composition of claim 21, wherein the one or more therapeutic agents are encapsulated within or conjucated to a lipid nanoparticle, nanostructured lipid carrier, a lipid drug conjugate-nanoparticle, a liposome, a transfersome, an ethosome, a liposphere, a niosome, a cubosome, a virosome, iscom, a nanoemulsion, or a phytosome.

23. The pharmaceutical composition of any one of claims 19-22, wherein the one or more complement inhibitors is an inhibitor of the enzymatic activity of a soluble complement protein.

24. The pharmaceutical composition of any one of claims 19-22, wherein the one or more complement inhibitors is an inhibitor of the cleavage of a complement component selected from the group consisting of: C5, C6, C7, C8, C9, factor D and factor B.

25. The pharmaceutical composition of any one of claims 19-22, wherein the one or more complement inhibitors is an inhibitor of the cleavage of C5.

26. The pharmaceutical composition of any one of claims 19-22, wherein the one or more complement inhibitors is a peptide, an antibody, a fusion protein, a small molecule, or an aptamer.

27. The pharmaceutical composition of any one of claims 19-22, wherein the one or more complement inhibitors is formulated for local administration in a patient in need thereof.

28. The pharmaceutical composition of claim 27, wherein the one or more complement inhibitors is formulated for administration at an extravascular location in a patient in need thereof.

29. The pharmaceutical composition of claim 27, wherein the one or more therapeutic agents are administered by an administration method selected from the group consisting of subcutaneous, intraperitoneal, intramuscular, intra-articular, intra-synovial, intrasternal, intrathecal, intrahepatic, intralesional, intracranial, intraventticular, oral, pulmonary, topical, rectal, nasal, buccal, vaginal, intratumoral and intradermal.

30. The pharmaceutical composition of claim 26, wherein, the one or more complement inhibitors is a peptide peptide or an antibody that binds to a soluble complement protein that is produced at said extravascular location.

31. The pharmaceutical composition of any one of claims 19-22, wherein the one or more complement inhibitors is provided in an amount sufficient to produce a clinically significant reduction in severity of at least one symptom of CARPA or CRS to a patient receiving treatment for a disease or disorder, as compared to when the one or more complement inhibitors is not provided with the one or more therapeutic agents.

32. The pharmaceutical composition of claim 31, wherein the clinically significant reduction in severity of at least one symptom of CARPA or CRS is resolved after a period of about 4 hours following administration to the patient.
